SEQUENCE LISTING (4 sequences)

SEQ ID NO: 1 - DNA, 1269 bp, SARS-CoV
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 2 - protein, 422 aa, SARS-CoV
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - protein, 18 aa, Artificial Sequence
  Partial peptide of N protein in SARS-CoV-2
GQTVTKKSAAEASKKPRC

SEQ ID NO: 4 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

(19) United States
(12) Patent Application Publication
(10) Pub. No.: US 2022/0373546 A1
(43) Pub. Date: Nov. 24, 2022

(54) METHODS AND REAGENTS OF IMMUNOLOGICALLY DETECTING SARS-CoV-2

(71) Applicant: FUJIREBIO INC., Tokyo (JP)

(72) Inventors: Masayasu IMAIZUMI, Tokyo (JP); Kentaro YAMAKAWA, Tokyo (JP); Tomonori NISHII, Tokyo (JP); Naoyuki TANAKA, Tokyo (JP); Azusa IIZUKA, Tokyo (JP); Akira FUJIMOTO, Tokyo (JP); Nobuyuki ISE, Tokyo (JP); Kazuyoshi MIYAMOTO, Tokyo (JP); Shintaro YAGI, Tokyo (JP); Katsumi AOYAGI, Tokyo (JP)

(73) Assignee: FUJIREBIO INC., Tokyo (JP)

(21) Appl. No.: 17/318,070

(22) Filed: May 12, 2021

(30) Foreign Application Priority Data
May 11, 2021 (JP) 2021-080408
May 11, 2021 (JP) 2021-080409

(51) Int. Cl. G01N 33/569; G01N 33/543

(52) U.S. Cl. CPC G01N 33/56983; G01N 33/543; G01N 2333/165

(57) ABSTRACT

The present invention provides a method of immunologically detecting SARS-CoV-2, the method comprising detecting a SARS-CoV-2 N protein in a specimen taken from a subject by using the following antibodies (1) and (2): (1) a first antibody to a first epitope in a 306th to 339th amino acid region in the SARS-CoV-2 N protein, and (2) a second antibody to a second epitope in a 365th to 419th amino acid region in the SARS-CoV-2 N protein.

### TECHNICAL FIELD

The present invention relates to methods and reagents of immunologically detecting SARS-CoV-2.

### BACKGROUND

The SARS coronavirus-2 (SARS-CoV-2) is classified into the genus betacoronavirus belonging to the family coronaviridae, and is the virus that causes COVID-19, a novel coronavirus infection disease. SARS-CoV-2 is closely related to but different from the SARS coronavirus (SARS-CoV) that causes a severe acute respiratory syndrome (SARS) prevailed in China in the 2000s.

The coronavirus is an envelope virus having a positive-sense single-stranded RNA as the virus genome. The nucleocapsid protein (N protein) in the virus particle forms a dimer by the C-terminal domain (CTD); this further forms a tetramer. It is believed that the genome RNA is bound to the N-terminal domain (NTD) of the nucleocapsid protein (N protein) to form the nucleocapsid. The nucleocapsid has the structure that is covered with an envelope formed of a lipid bilayer membrane. The S protein, E protein, and M protein are bound to the envelope in the transmembrane domain thereof. The N protein is bound to the M protein that constitutes the envelope to form the virus particle covered with the envelope.

There is no report about the molecular form of the N protein in the specimen. It is generally believed that there are (i) the one that is included in the nucleocapsid of the virus particle and (ii) the one that is released from the cell that is infected with the virus and then destructed due to proliferation of the virus or due to an immunological reaction of the infected host.

As for the immunological measurement method of the SARS coronavirus, there is a report about the method which uses an antibody to the SARS-CoV N protein (total length: 422 amino acids).

Patent Literature 1 describes various monoclonal antibodies to the SARS-CoV N protein and immunological measurement reagents using these antibodies. Disclosed therein was also the immunological measurement reagent that is used in the sandwich method which uses two or more antibodies of a solid phase antibody and a labelled antibody each capable of recognizing a different epitope. Patent Literature 1 also states that both the solid phase antibody and the labelled antibody included in the immunological measurement reagent may be selected from two or more monoclonal antibodies, and then, they can be used as a combination.

Patent Literature 2 describes a measurement method of the SARS-CoV N protein by using a first antibody and a second antibody that specifically bind to the SARS-CoV N protein; in this method, the first antibody or the second antibody is the antibody that recognizes the epitope present in the region of the 283rd to 422nd from the N-terminal of the amino acid sequence of the N protein (region C). Patent Literature 2 states that the SARS-CoV N protein (1 to 422 amino acids) is divided into three regions (region A: the 1st to 141st region, region B: the 142nd to 282nd region, and region C: the 283rd to 422nd region), and then that the SARS-CoV N protein can be measured with a higher sensitivity than ever by using a combination of (a) the antibody to the 283rd to 422nd region and (b) the antibody to the 1st to 141st region or to the 142nd to 282nd region.

In connection with the immunological measurement method of the SARS coronavirus, there is a report of the method in which the specimen that include SARS-CoV is treated with a nonionic surfactant (for example, Triton X100 or NP40) or an anionic surfactant (for example, SDS) (Patent Literatures 3 and 4).

### PRIOR ART LITERATURES

### Patent Literatures

Patent Literature 1: International Publication No. WO2005/042579

Patent Literature 2: International Publication No. WO2007/043582

Patent Literature 3: Japanese Patent Application Laid-open No. 2009-109426

Patent Literature 4: International Publication No. WO2005/042579

### SUMMARY OF THE INVENTION

### Problem to be Solved by the Invention

The object of the present invention is to provide a technology that can immunologically detect SARS-CoV-2.

### Means for Solving Problem

The inventors of the present invention carried out an extensive investigation; as a result, it was found that SARS-CoV-2 can be detected with high sensitivity by using a combination of specific two antibodies to two epitopes in the C-terminal region in the SARS-CoV-2 N protein, that is, by using a combination of (1) a first antibody to a first epitope in the 260th to 305th amino acid region in the SARS-CoV-2 N protein and (2) a second antibody to a second epitope in the 365th to 419th amino acid region in the SARS-CoV-2 N protein. The inventors of the present invention also found not only that these antibodies can be used easily as a combination with other antibody, but also that the combination with other antibody is a useful combination of the antibodies that can realize detection of SARS-CoV-2 with a far higher sensitivity.

Patent Literatures 1 and 2 are the technology enabling to immunologically detect SARS-CoV by using the antibody to the SARS-CoV N protein (the total amino acid length thereof is reported 422), but does not disclose the immunological detection of SARS-CoV-2 by using the antibody to the SARS-CoV-2 N protein (the total amino acid length thereof is reported 419). In addition, Patent Literature 1 does not disclose even the epitope in the N protein to be targeted in the immunological detection of SARS-CoV that is the virus different from SARS-CoV-2. Patent Literature 2 describes, in the immunological detection of SARS-CoV, use of a combination of two specific antibodies to two epitopes in the different regions divided into three as mentioned in (a) and (b), but this neither teaches nor suggests that the combination of two specific antibodies to two epitopes in the C-terminal region in the SARS-CoV-2 N protein is superior in detection of SARS-CoV-2.

The inventors of the present invention further carried cut an extensive investigation; as a result, it was also found that when the specimen was mixed with a zwitterionic surfactant comprising a hydrocarbon chain with the chain length of 12 to 18 carbon atoms, SARS-CoV-2 could be immunologically detected with high sensitivity.

Patent Literatures 3 and 4 are the technology enabling to immunologically detect SARS-CoV, but do not disclose that SARS-CoV-2 can be immunologically detected with high sensitivity. In addition, Patent Literatures 3 and 4 neither teach nor suggest the use of the zwitterionic surfactant in the immunological detection.

On the basis of the findings described above, the inventors of the present invention succeeded to develop the method and reagent for immunological detection of SARS-CoV-2; as a result, the present invention could be completed.

Namely, the present invention is as follows.

[1] A method of immunologically detecting SARS-CoV-2, the method comprising detecting a SARS-CoV-2 nucleocapsid protein (N protein) in a specimen taken from a subject by using the following antibodies (1) and (2):
(1) a first antibody to a first epitope in a 260th to 305th amino acid region in the SARS-CoV-2 N protein; and
(2) a second antibody to a second epitope in a 365th to 419th amino acid region in the SARS-CoV-2 N protein.
   [2] The method according to [1], wherein a third antibody to a third epitope in a 120th to 147th amino acid region in the SARS-CoV-2 N protein is further used.
   [3] The method according to [1] or [2], wherein a fourth antibody to a fourth epitope in a 44th to 78th amino acid region in the SARS-CoV-2 N protein is further used.
   [4] The method according to any of [1] to [3], wherein a fifth antibody to a fifth epitope in a 243rd to 259th amino acid region in the SARS-CoV-2 N protein is further used.
   [5] The method according to any of [1] to [4], wherein a sixth antibody to a sixth epitope in a 306th to 339th amino acid region in the SARS-CoV-2 N protein is further used.
   [6] The method according to any of [1] to [5], wherein both the first antibody and the second antibody are used as a solid phase antibody or a labelled antibody.
   [7] The method according to [6], wherein
   when both the first antibody and the second antibody are used as the solid phase antibody, the third antibody is used as the labelled antibody, and
   when both the first antibody and the second antibody are used as the labelled antibody, the third antibody is used as the solid phase antibody.
      [8] The method according to [6] or [7], wherein
   the fourth antibody is used as the solid phase antibody and the fifth antibody is used as the labelled antibody, or
   the fourth antibody is used as the labelled antibody and the fifth antibody is used as the solid phase antibody.
      [9] The method according to any of [1] to [8], wherein the detection is carried out by a sandwich method.
      [10] A reagent of immunologically detecting SARS-CoV-2, the reagent comprising the following antibodies (1) and (2):
(1) a first antibody to a first epitope in a 260th to 305th amino acid region in a SARS-CoV-2 N protein; and
(2) a second antibody to a second epitope in a 365th to 419th amino acid region in the SARS-CoV-2 N protein.
   [11] The reagent according to [10], the reagent further comprising one or more antibodies selected from the group consisting of the following (3) to (6):
(3) a third antibody to a third epitope in a 120th to 147th amino acid region in the SARS-CoV-2 N protein;
(4) a fourth antibody to a fourth epitope in a 44th to 78th amino acid region in the SARS-CoV-2 N protein;
(5) a fifth antibody to a fifth epitope in a 243rd to 259th amino acid region in the SARS-CoV-2 N protein; and
(6) a sixth antibody to a sixth epitope in a 306th to 339th amino acid region in the SARS-CoV-2 N protein.
   [12] The reagent according to [10] or [11], wherein both the first antibody and the second antibody are a solid phase antibody or a labelled antibody.
   [13] The reagent according to any of [10] to [12], wherein the reagent is used in a sandwich method.
   [14] The reagent according to any of [11] to [13], wherein the reagent comprises a solid phase.

In addition, the present invention is as follows.

[1] A method of immunologically detecting SARS-CoV-2, wherein a specimen taken from a subject is mixed with a solution containing a zwitterionic surfactant comprising a hydrocarbon chain with the chain length of 12 to 18 carbon atoms.
[2] The method according to [1], wherein the hydrocarbon chain has the chain length of 14 to 18 carbon atoms.
[3] The method according to [1] or [2], wherein the hydrocarbon chain is an alkyl chain.
[4] The method according to any one of [1] to [3], wherein the zwitterionic surfactant is a zwitterionic surfactant comprising an ammonium group having the hydrocarbon chain.
[5] The method according to any one of [1] to [4], wherein the zwitterionic surfactant comprising the ammonium group having the hydrocarbon chain is 3-(N,N-dimethyldodecylammonio) propanesulfonate (C12APS), 3-(N,N-dimethylmyristylammonio) propanesulfonate (C14APS), 3-(N,N-dimethylpalmitylammonio) propanesulfonate (C16APS), or 3-(N,N-dimethylstearylammonio) propanesulfonate (C18APS).
[6] The method according to any one of [1] to [5], wherein the solution further includes an anionic surfactant or zwitterionic surfactant having a steroid skeleton.
[7] The method according to [6], wherein the anionic surfactant or zwitterionic surfactant having the steroid skeleton is a bile acid or a derivative thereof retaining the steroid skeleton, or a salt thereof.
[8] The method according to [7], wherein the bile acid or the derivative thereof retaining the steroid skeleton is one or more compounds selected from the group consisting of deoxycholic acid, chenodeoxycholic acid, ursodeoxycholic acid, hyodeoxycholic acid, cholic acid, glycocholic acid, taurocholic acid, hyocholic acid, 5α-cyprinol, lithocholic acid, taurodeoxycholic acid, taurocholic acid, CHAPS, and CHAPSO.
[9] The method according to any one of [1] to [8], wherein the solution further includes a nonionic surfactant.
[10] The method according to any one of [1] to [9], wherein one or more antibodies to a SARS-CoV-2 nucleocapsid protein (N protein) are used in the immunological detection.
[11] The method according to any one of [1] to [10], wherein the immunological detection is carried out by a sandwich method.
[12] A reagent of immunologically detecting SARS-CoV-2, wherein the reagent includes the following components (1) and (2):
(1) a zwitterionic surfactant comprising a hydrocarbon chain with a chain length of 12 to 18 carbon atoms; and
(2) one or more antibodies to a target molecule that constitutes SARS-CoV-2.
   [13] The reagent according to [12], wherein the reagent further includes the following component(s) (3) and/or (4):
(3) an anionic surfactant or zwitterionic surfactant having a steroid skeleton; and/or
(4) a nonionic surfactant.
   [14] The reagent according to [12] or [13], wherein the reagent is a reagent that is used in a sandwich method.

### Effect of the Invention

According to the present invention, SARS-CoV-2 can be detected with high sensitivity.

### BRIEF DESCRIPTION OF DRAWINGS

FIG. 1 is a conceptional drawing illustrating one example of the immunochromatographic cartridge.

FIG. 2 illustrates (A) amino acid sequence of SARS-CoV N protein (SEQ ID NO:1), and (B) amino acid sequence of SARS-CoV-2 N protein (SEQ ID NO:4).

### EMBODIMENTS FOR CARRYING OUT THE INVENTION

### 1. Invention Relating to Combination of Antibodies

The present invention provides a method of immunologically detecting SARS-CoV-2, the method including detecting a SARS-CoV-2 N protein in a specimen taken from a subject by using the following antibodies (1) and (2):
(1) a first antibody to a first epitope in a 260th to 305th amino acid region in the SARS-CoV-2 N protein; and
(2) a second antibody to a second epitope in a 365th to 419th amino acid region in the SARS-CoV-2 N protein.

For a subject from which a specimen is available, any subject that may be infected with SARS-CoV-2 may be used. Illustrative examples of such a subject include mammals (for example, primates such as human and monkey; rodents such as mouse, rat, and rabbit; ungulates such as cow, pig, goat, horse, and sheep; and carnivorous animals such as dog and cat) and birds (for example, chicken). Preferably, the subject is a mammal such as human. In view of clinical application, the subject is preferably human.

For a specimen, any biological sample which may include SARS-CoV-2 may be used. Illustrative examples of such a specimen include saliva, sputum, nasal mucous, swab (swabs of mucous membrane portions such as nasal swab and oropharyngeal swab), lavage fluids (for example, nasal lavage fluid, oral lavage fluid, bronchial lavage fluid, and lung lavage fluid), bloods (for example, whole blood, plasma, and serum), feces, as well as other specimen (for example, specimen comprising infected cells). From a viewpoint of low invasiveness, as well as prompt and easy access to the specimen which may include large quantity of SARS-CoV-2, the specimen is preferably saliva, sputum, nasal mucous, or swab. The specimen may be subjected to a treatment in advance. Illustrative examples of the treatment include centrifugal separation, extraction, dilution, filtration, precipitation, heating, freezing, refrigerating, stirring, as well as treatment with a component such as a surfactant.

The SARS-CoV-2 N protein to be detected in the present invention is an N protein in any strain of SARS-CoV-2 (namely, a wild type or a mutant type N protein). Illustrative examples of the strain include main strains of L-type and S-type, as well as sub-strains thereof. Many strains of SARS-CoV-2 are reported. For these strains, for example, in the influenza virus gene data base GISAID (Global Initiative on Sharing All Influenza Data) may be referred to. The genome sequence information of SARS-CoV-2 has been disclosed (see, e.g., GenBank accession No. MN908947 for the Wuhan-Hu-1 strain of SARS-CoV-2). Therefore, for the SARS-CoV-2 N protein, the N protein encoded by such a genome sequence may be referred to. Also, for the wild type N protein of SARS-CoV-2, the amino acid sequence of SEQ ID NO:4 may be referred to.

In the present invention, the SARS-CoV-2 N protein may be detected by using the following antibodies (1) and (2):
(1) a first antibody to a first epitope in a 260th to 305th amino acid region in the SARS-CoV-2 N protein; and
(2) a second antibody to a second epitope in a 365th to 419th amino acid region in the SARS-CoV-2 N protein.

The first epitope is present in the 260th to 305th amino acid region in the SARS-CoV-2 N protein.

The second epitope is present in the 365th to 419th amino acid region in the SARS-CoV-2 N protein.

In one embodiment, the method of the present invention may further include use of a third antibody to a third epitope in a 120th to 147th amino acid region in the SARS-CoV-2 N protein. When the third antibody is used as a combination together with the antibodies (1) and (2), the SARS-CoV-2 N protein can be detected with high sensitivity. The third epitope is present in the 120th to 147th amino acid region in the SARS-CoV-2 N protein.

In another embodiment, the method of the present invention may further include use of a fourth antibody to a fourth epitope in a 44th to 78th amino acid region in the SARS-CoV-2 N protein. When the fourth antibody is used as a combination together with the antibodies (1) and (2), the SARS-CoV-2 N protein can be detected with high sensitivity. The fourth epitope is present in the 44th to 78th amino acid region in the SARS-CoV-2 N protein.

In still another embodiment, the method of the present invention may further include use of a fifth antibody to a fifth epitope in a 243rd to 259th amino acid region in the SARS-CoV-2 N protein. When the fifth antibody is used as a combination together with the antibodies (1) and (2), the SARS-CoV-2 N protein can be detected with high sensitivity. The fifth epitope is present in the 243rd to 259th amino acid region in the SARS-CoV-2 N protein.

In still another embodiment, the method of the present invention may further include use of a sixth antibody to a sixth epitope in a 306th to 339th amino acid region in the SARS-CoV-2 N protein. When the sixth antibody is used as a combination together with the antibodies (1) and (2), the SARS-CoV-2 N protein can be detected with high sensitivity. The sixth epitope is present in the 306th to 339th amino acid region in the SARS-CoV-2 N protein.

In addition, the present invention provides the method of immunologically detecting SARS-CoV-2, the method including detecting the SARS-CoV-2 N protein in the specimen taken from a subject by using the following combinations of the antibodies:
(1) a combination of the third antibody with the first antibody;
(2) a combination of the third antibody with the second antibody;
(3) a combination of the fourth antibody with the first antibody;
(4) a combination of the fourth antibody with the second antibody;
(5) a combination of the sixth antibody with the first antibody;
(6) a combination of the fifth antibody with the first antibody; or
(7) a combination of the fifth antibody with the third antibody.

The antibody may be any of a polyclonal antibody and a monoclonal antibody. The antibody may be any isotype of immunoglobulins (for example, IgG, IgM, IgA, IgD, IgE, and IgY). The antibody may also be a full-length antibody. The full-length antibody means an antibody comprising a heavy chain and a light chain, each comprising both a variable region and a constant region (for example, an antibody comprising two Fab portion and a Fc portion). The antibody may also be an antibody fragment originated from the whole length antibody. The antibody fragment is part of the full-length antibody; examples thereof include a constant region deletion antibody (such as F(ab′)₂, Fab′, Fab, and Fv). The antibody may also be a modified antibody such as a single-chain antibody.

The antibody may be prepared by heretofore known methods. For example, the antibody may be efficiently prepared by using the above-mentioned epitopes as the antigen.

In one embodiment, the antibody may be a solid phase antibody. The solid phase antibody is the antibody that is immobilized to a solid phase. Illustrative examples of the solid phase include a solid phase which can be suspended or dispersed in a liquid phase (for example, solid phase carriers such as particles and beads) and a solid phase which can accommodate or carry a liquid phase (for example, supports such as a plate, a membrane, and a test tube; and containers such as a well plate, a micro flow channel, a glass capillary, a nanopillar, and a monolith column). Illustrative examples of the material of the solid phase include a glass, a silica, a polymer compound (for example, polystyrene and plastics), a metal, and a carbon. Also, a non-magnetic material or a magnetic material may be used as the material of the solid phase. The antibody may be immobilized to the solid phase by any method. Illustrative examples of the method include a covalent bonding method, a method using an affinity substance (for example, biotin and streptavidin), an ionic bonding method, and a physically adsorbing method. In the covalent bonding method, periodic acid, glutaraldehyde, maleimide, N-hydroxy succinimide, or the like may be used.

In another embodiment, the antibody may be a labelled antibody. The labelled antibody means an antibody that is labelled with a labelling substance. Illustrative examples of the labelling substance include enzymes (for example, peroxidase, alkaline phosphatase, luciferase, and β-galactosidase), affinity substances (for example, any one of streptavidin and biotin, and any one of a sense strand and an antisense strand which are complementary each other in nucleic acids), fluorescent substances (for example, fluorescein, fluorescein isothiocyanate, rhodamine, green fluorescent protein, and red fluorescent protein), luminescent substances (for example, luciferin, aequorin, acridinium ester, tris(2,2′-bipyridyl)ruthenium, and luminol), radioactive substances (for example, ³H, ¹⁴C, ³²P, ³⁵S, and ¹²⁵I) , metal colloids (for example, gold colloid, silver colloid, platinum colloid, iron oxide colloid, and aluminum hydroxide colloid), and colored substances (for example, latex particles colored with a colorant, a dye, or a pigment). Labelling of the antibody with the labelling substance may be carried out by any method. Examples of the method include the methods as mentioned in immobilization of the antibody to the solid phase.

In a certain embodiment, both the first antibody and the second antibody may be used as the solid phase antibody or as the labelled antibody. When both the first antibody and the second antibody are used together as the solid phase antibody or as the labelled antibody, there are merits that the SARS-CoV-2 N protein can be detected with a far higher sensitivity, and that the bonding competition between the solid phase antibody and the labelled antibody, which takes place frequently, can be avoided readily. More preferably, both the first antibody and the second antibody may be used as the solid phase antibody.

In another specific embodiment, the third antibody may be used as the labelled antibody or as the solid phase antibody. More specifically, when both the first antibody and the second antibody are used as the solid phase antibody, the third antibody may be used as the labelled antibody. When both the first antibody and the second antibody are used as the labelled antibody, the third antibody may be used as the solid phase antibody. When the third antibody is used in the form that is different from the first antibody or from the second antibody, the SARS-CoV-2 N protein can be detected with a far higher sensitivity.

In another specific embodiment, the fourth antibody and the fifth antibody may be used in different forms with each other. More specifically, when the fourth antibody is used as the solid phase antibody, the fifth antibody may be used as the labelled antibody. When the fourth antibody is used as the labelled antibody, the fifth antibody may be used as the solid phase antibody. When the fourth antibody and the fifth antibody are used in different forms with each other, the SARS-CoV-2 N protein can be detected with a far higher sensitivity.

In still another specific embodiment, the sixth antibody may be used as the labelled antibody or as the solid phase antibody.

In the present invention, the SARS-CoV-2 N protein can be detected by the immunological method using the above-mentioned antibodies. Illustrative examples of the immunological method include a direct competitive method, an indirect competitive method, a sandwich method, a Western blotting method, and an immunohistochemical staining method. Preferably, the immunoassay may be the sandwich method. Illustrative examples of the immunoassay include chemiluminescent immunoassay (CLIA) (for example, chemiluminescent enzyme immunoassay (CLEIA)), turbidimetric immunoassay (TIA), enzyme immunoassay (EIA) (for example, direct competitive ELISA method, indirect competitive ELISA method, and a sandwich ELISA method), a radioimmunoassay (RIA), a latex agglutination reaction method, fluorescent immunoassay (FIA), and an immunochromatographic method. Here, the method can be used so far as the measurement system is based on the principle that bonding between the antigen and the antibody can be specifically detected. The method of the present invention may be a qualitative method or a quantitative method.

The label may be detected with an appropriately selected method in accordance with the label. For example, when the label is an enzyme, the label may be detected by detecting the enzyme activity using a signal generating substrate (for example, a fluorescent substrate, a luminescent substrate, and a chromogenic substrate). When the label is an affinity substance, by using an enzyme or the signal generating substance that is capable of bonding to the affinity substance, the label may be detected by detecting the enzyme or the signal generating substance that is bonded to the affinity substance. The enzyme or the signal generating substance that is capable of bonding to the affinity substance may be the enzyme or the signal generating substance that is bonded to the substance that is capable of bonding to the affinity substance. When the label is a fluorescent substance, a luminescent substance, or a radioactive substance, the label may be detected by detecting the signal that is generated from these labels. When the chromogenic substance is used as the labelling substance, the label may be detected with a visual observation.

In a specific embodiment, the method of the present invention may be carried out by the sandwich method. The sandwich method is superior in the sensitivity and the specificity.

In the sandwich method, detection of the N protein may be carried out, for example, with the process at which the N protein in the specimen is bonded to the solid phase antibody by treating the specimen with the solid phase antibody (for example, the process at which the specimen is contacted with the solid phase antibody) and the process at which the N protein that is bound to the solid phase antibody is detected by the labelled antibody. The detection of the N protein may further include the process at which the N protein that is not bound to the solid phase antibody is removed (for example, B/F separation or washing process). Also, in the sandwich method, detection of the N protein may be carried out, for example, with the process at which the N protein in the specimen is bound to the labelled antibody by treating the specimen with the labelled antibody (for example, the process at which the specimen is contacted with the labelled antibody), the process at which the N protein is bound to the solid phase antibody by further treating the N protein to the solid phase antibody, and the process at which the N protein bound to the solid phase antibody and to the labelled antibody are detected by the labelled antibody. The antibody that has been immobilized to the solid phase in advance or the antibody that is immobilized to the solid phase during the time of detecting the N protein may be used as the solid phase antibody. The detection may be carried out, for example, with the process at which the N protein in the specimen is bound to the antibody by treating the specimen with the antibody to be immobilized to the solid phase (for example, the process at which the specimen is contacted with the solid phase antibody), the process at which the N protein that is bound to the antibody to be immobilized to the solid phase is bound to the labelled antibody, the process at which the antibody to be immobilized to the solid phase is immobilized to the solid phase, and the process at which the N protein that is bound to the antibody that is immobilized to the solid phase (solid phase antibody) and the labelled antibody are detected by the labelled antibody. In the method of binding the antibody to be immobilized to the solid phase to the solid phase, the above-mentioned affinity substances may be used.

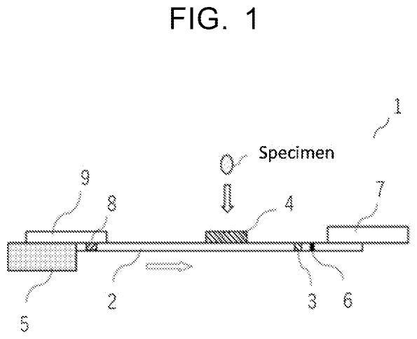

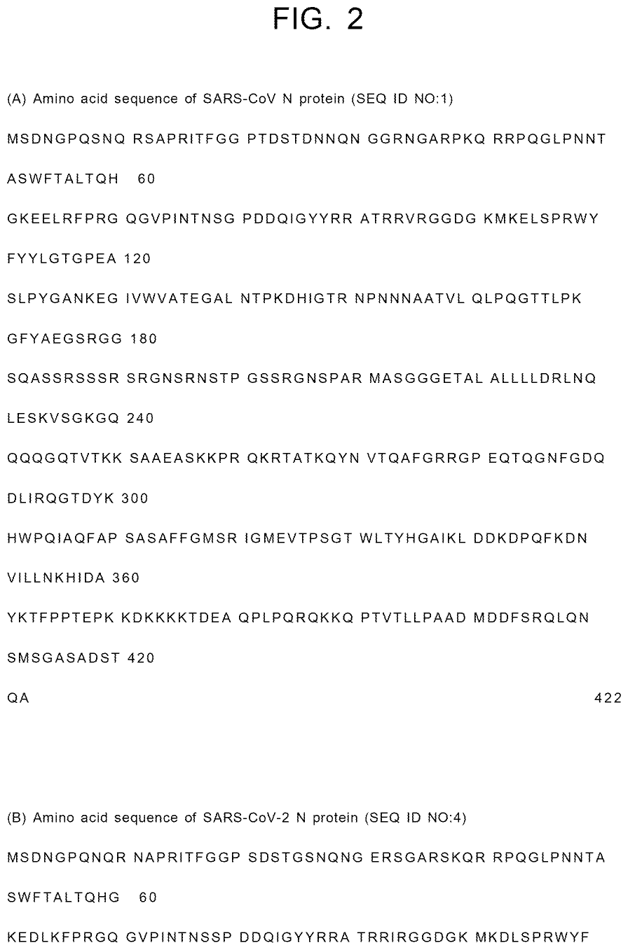

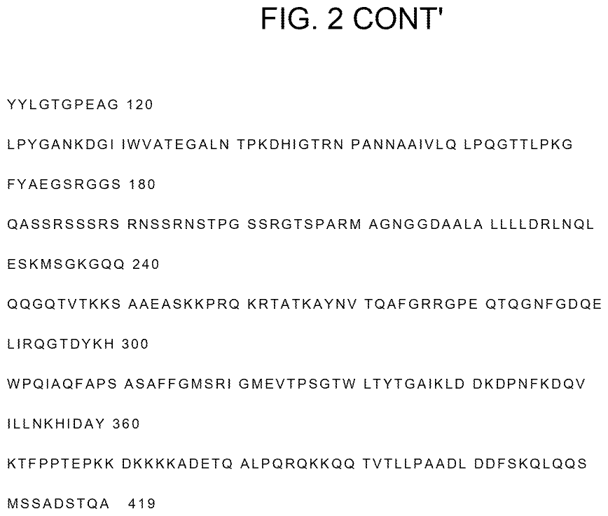

In another specific embodiment, the method of the present invention may be carried out by an immunochromatographic method. The immunochromatographic method is preferable as a prompt and convenient qualitative method. The immunochromatographic method and the apparatuses that are used in this method are well known. One example of the usable apparatus is the immunochromatographic cartridge having the zone that includes a solid phase antibody and the zone that includes a labelled antibody (this may further have a storage member for a developing solution). Hereinafter, an outline of a lateral flow type immunochromatographic method, which is one preferable example of the immunochromatographic method, and the apparatus thereof will be explained with referring to FIG. 1.

In FIG. 1, an immunochromatographic cartridge 1 has, on a matrix 2 formed of a porous material such as a nitrocellulose membrane, a detecting zone 3 in which a capturing antibody is solid-phased in a line (zone that includes a solid phase antibody), and in the upstream side thereof (upstream in the flow direction of a developing solution to be described later), a labelled reagent zone 4 that supports a detecting antibody (zone that includes a labelled antibody). Usually, the matrix 2 is formed in a strip form. The labelled reagent zone 4 is formed of a porous pad to which the labelled antibody is spotted. In the upstream end of the matrix, a developing solution tank 5 in which a developing solution is stored is arranged. In the downstream of the detecting zone, a development confirming part 6 to confirm that the developing solution has flowed, and in the further downstream thereof, a developing solution-absorbing zone 7 in which a porous absorbing pad to absorb the developing solution is arranged. In the development confirming part, a probe (for example, antibody) having an affinity with the substances other than the detection substance (N protein) that flows with the developing solution, such as an anti-label antibody, is solid-phased in a line. When the label is an enzyme, in the upstream of the labelled reagent zone, a substrate zone 8 to which a substrate of the enzyme is supported is arranged. In the vicinity of the developing solution tank 5, a member such as a pressing member having a projection (not illustrated in the figure) that can break the developing solution tank by pressing the projection thereto is arranged in advance so that the developing solution tank may be readily broken to feed the developing solution to the matrix. In addition, a developing solution pad 9 may be arranged in such a way as to cover the developing solution tank and the upper end of the matrix in order to help feeding of the developing solution in the broken developing solution tank to the upper end of the matrix.

When using, the specimen is added to the labelled reagent zone 4; then, the developing solution tank 5 is broken to cause the developing solution contact to the upper end of the matrix thereby starting the feeding. The developing solution thereby fed to the matrix flows toward the downstream due to the capillary phenomenon of the matrix. When the developing solution passes through the substrate zone, the substrate is eluted into the developing solution; and thus the developing solution comprising the substrate flows. Next, when the developing solution passes through the labelled reagent zone, the labelled antibody and the specimen are eluted into the developing solution; and thus the developing solution comprising the substrate, the labelled antibody, and the specimen flows. When the N protein is included in the specimen, the N protein and the labelled antibody bound with each other by the antigen-antibody reaction. When they reach the detecting zone, the solid phase antibody and the N protein bound on the detecting zone by the antigen-antibody reaction. As a result, the labelled antibody is immobilized to the detecting zone by intervention of the N protein. Then, by detecting the label that is immobilized to the detecting zone, the N protein is detected. When the N protein is not included in the specimen, nothing is bound to the solid-phased antibody; and thus the labelled antibody is not immobilized to the detecting zone. The developing solution further flows toward the downstream to reach the developing solution confirming part. Here, for example, when the anti-label antibody is immobilized to the developing solution confirming part, the labelled antibody that has not been bound to the N protein is bound to the anti-label antibody by the antigen-antibody reaction; as a result, the labelled antibody is immobilized to the developing solution confirming part. When the label is detected in the developing solution confirming part, it can be confirmed that the developing solution has been correctly developed till the development confirming part. Thereafter, the developing solution is absorbed to the absorbing pad located in the further downstream.

In addition, the present invention provides a reagent of immunologically detecting SARS-CoV-2 including the following antibodies (1) and (2):
(1) a first antibody to a first epitope in a 260th to 305th amino acid region in the SARS-CoV-2 N protein; and
(2) a second antibody to a second epitope in a 365th to 419th amino acid region in the SARS-CoV-2 N protein.

The reagent of the present invention may further include one or more antibodies selected from the group consisting of the following (3) to (6):
(3) a third antibody to a third epitope in a 120th to 147th amino acid region in the SARS-CoV-2 N protein;
(4) a fourth antibody to a fourth epitope in a 44th to 78th amino acid region in the SARS-CoV-2 N protein;
(5) a fifth antibody to a fifth epitope in a 243rd to 259th amino acid region in the SARS-CoV-2 N protein; and
(6) a sixth antibody to a sixth epitope in a 306th to 339th amino acid region in the SARS-CoV-2 N protein.

In addition, the present invention provides a reagent of immunologically detecting SARS-CoV-2, including the following combinations of the antibodies:
(1) a combination of the third antibody with the first antibody;
(2) a combination of the third antibody with the second antibody;
(3) a combination of the fourth antibody with the first antibody;
(4) a combination of the fourth antibody with the second antibody;
(5) a combination of the sixth antibody with the first antibody;
(6) a combination of the fifth antibody with the first antibody; or
(7) a combination of the fifth antibody with the third antibody.

Details of the terms of the above-mentioned antibodies and the like in the reagent of the present invention (for example, definitions, examples, and preferred examples) are the same as those described in the method of the present invention.

The reagent of the present invention may have the first antibody and the second antibody in the mixed form (for example, in the stored form in the same container, or in the form of solid phase antibodies immobilized to the same solid phase), or in the separated form to each other (for example, in the stored form in different containers). In addition, the reagent of the present invention may include, in addition to the first antibody and the second antibody, one or more antibodies selected from the group consisting of the above (3) to (6) in the mixed form or in the separated form to each other. The reagent of the present invention may be provided in the form of a kit as well. The reagent of the present invention may also be provided in the form of a device. Specifically, all the antibodies may be accommodated in the device. Alternatively, part of the antibodies may be accommodated in the device while the rest of the antibodies is not necessarily accommodated in the device (for example, in the form stored in a different container). In this case, the antibody not accommodated in the device may be charged into the device at the time of detection.

The reagent of the present invention may include the solid phase antibody and/or the labelled antibody. Alternatively, when the reagent does not include the solid phase antibody and/or the labelled antibody, the reagent may include a solid phase and/or a labelling substance. The solid phase and the labelling substance are the same as those described before. When the labelling substance is an enzyme, the reagent of the present invention may include a substrate of the enzyme (for example, the substrate that generates a detection signal, or the substrate that is transformed to a product generating a detection signal by the enzyme, or the substrate in the reaction capable of conjugating with other enzyme reaction using the substrate that generates a detection signal or the substrate that is transformed to a product generating a detection signal by the enzyme).

The reagent of the present invention is not particularly restricted so far as this can be used in the immunological measurement method; and thus the reagent can have a composition in accordance with the immunological measurement method. Preferably, the reagent of the present invention may be the reagent that is used in the sandwich method. Therefore, the reagent of the present invention may include a solid phase that is used in the sandwich method (for example, particles such as magnetic particle, a well plate, and a membrane), or a solid phase antibody immobilized to the above-mentioned solid phase. Also, the reagent of the present invention may include a device including the solid phase that is used in the immunochromatographic method (namely, the above-mentioned immunochromatographic cartridge), or the solid phase antibody that is immobilized to the above-mentioned solid phase. The reagent of the present invention may include a N protein standard.

### 2. Invention Relating to Specimen Treatment

The present invention provides a method of immunologically detecting SARS-CoV-2, wherein a specimen taken from a subject is mixed with a solution comprising a zwitterionic surfactant comprising a hydrocarbon chain with the chain length of 12 to 18 carbon atoms.

The specimen and the subject are the same as those described before.

For a solution to be mixed with the specimen, an aqueous solution may be used. Illustrative examples of the aqueous solution include water (for example, distilled water, sterilized water, sterilized distilled water, and purified water) and a buffer. The buffer is preferable. Illustrative examples of the buffer include a phosphate buffer, a phosphate-buffered physiological saline (PBS), an tartrate buffer, a citrate buffer, an acetate buffer, a glycine buffer, a carbonate buffer, a 2-morpholino ethanesulfonate (MES) buffer, a trishydroxymethyl aminomethane (Tris) buffer, a borate buffer, a 3-morpholino propanesulfonate (MOPS) buffer, an N,N-bis(2-hydroxyethyl)glycine (Bicine) buffer, a bis(2-hydroxyethyl)iminotris(hydroxymethyl)methane (Bis-Tris) buffer, a 2-[4-(2-hydroxyethyl)1-piperadinyl ethanesulfonate (HEPES) buffer, and an imidazole buffer. The solution may include a small amount of an organic solvent (for example, an alcohol). The volume of the solution may be determined as appropriate in accordance with the factors such as the volume of the specimen. The volume may be, for example, in the range of 1 μL to 10 mL, and preferably in the range of 5 μL to 5 mL, while more preferably in the range of 10 μL to 1 mL. Preferably, pH of the buffer is neutral. More specifically, pH may be preferably 5.0 or more, and more preferably 5.5 or more, while still more preferably 6.0 or more. Also, pH may be preferably 9.0 or less, and more preferably 8.5 or less, while still more preferably 8.0 or less. Measurement of pH may be done by using a heretofore known method in this field. Preferably, the value that is obtained by measurement using a pH meter equipped with a glass electrode at 25° C. may be used as the pH value.

The zwitterionic surfactant has the hydrocarbon chain with the chain length of 12 to 18 carbon atoms. Preferably, the carbon atom number may be 13 or more, 14 or more, 15 or more, or 16 or more. Also, the carbon atom number may be 17 or less, or 16 or less. The hydrocarbon chain may be a linear chain or a branched chain, and preferably a linear chain. The hydrocarbon chain may be an alkyl chain, an alkenyl chain, or an alkynyl chain, and preferably an alkyl chain. By mixing such a zwitterionic surfactant with the specimen, the protein in the specimen can be immunologically detected with high sensitivity. Only one kind of the zwitterionic surfactant may be used, or two or more kinds thereof may be used concurrently.

The zwitterionic surfactant comprising the hydrocarbon chain also has both a cationic portion (for example, ammonium, phosphonium, and sulfonium) and an anionic portion (for example, a sulfate group, a sulfonate group, a carboxylate group, and a phosphate group). Accordingly, in the present invention, the surfactant comprising the hydrocarbon chain with the chain length of 12 to 18 carbon atoms, as well as the cationic portion and the anionic portion can be used.

The zwitterionic surfactant comprising the hydrocarbon chain may be in a free form or in a salt form. Illustrative examples of the salt form include metal salts (for example, monovalent metal salts such as sodium salts and potassium salts; and divalent metal salts such as calcium salts and magnesium salts), inorganic salts (for example, halide salts such as fluorides, chlorides, bromides, iodides, as well as ammonium salts), organic salts (for example, ammonium salts substituted with an alkyl group), and acid-added salts (for example, salts of inorganic acids such as sulfuric acid, hydrochloric acid, hydrobromic acid, nitric acid, and phosphoric acid; and salts of organic acids such as acetic acid, oxalic acid, lactic acid, citric acid, trifluoromethanesulfonic acid, and trifluoroacetic acid).

In a specific embodiment, the zwitterionic surfactant comprising the hydrocarbon chain may include an ammonium group having the hydrocarbon chain. Illustrative examples of such a zwitterionic surfactant include a surfactant comprising both a quaternary ammonium having the hydrocarbon chain and a sulfonate, and a surfactant comprising both a quaternary ammonium having the hydrocarbon chain and a carboxylate.

Illustrative examples of the surfactant comprising both a quaternary ammonium having the hydrocarbon chain and a sulfonate include 3-(N,N-dimethyldodecylammonio) propanesulfonate (C12APS), 3-(N,N-dimethylmyristylammonio) propanesulfonate (C14APS), 3-(N,N-dimethylpalmitylammonio) propanesulfonate (C16APS), 3-(N,N-dimethylstearylammonio) propanesulfonate (C18APS), and salts thereof.

Illustrative examples of the surfactant comprising both a quaternary ammonium having the hydrocarbon chain and a carboxylate include C12 to C18 alkyl dimethylaminoacetate betaines such as lauryl (C12) dimethylaminoacetate betaine, myristyl (C14) dimethylaminoacetate betaine, palmityl (C16) dimethylaminoacetate betaine, stearyl (C18) dimethylaminoacetate betaine, and salts thereof.

The zwitterionic surfactant comprising the hydrocarbon chain is preferably the surfactant comprising a quaternary ammonium having the hydrocarbon chain and a sulfonate.

The concentration of the zwitterionic surfactant comprising the hydrocarbon chain (final concentration in the mixed solution comprising the specimen) is not particularly restricted so far as the concentration thereof is enough for mixing with the specimen. Although the concentration changes in accordance with the factors such as the specimen and the target molecule to be immunologically detected (for example, target proteins such as the N protein), the concentration may be, for example, in the range of 0.001 to 10% by weight, preferably in the range of 0.005 to 5% by weight, more preferably in the range of 0.01 to 2% by weight, still more preferably in the range of 0.02 to 1.8% by weight, and particularly preferably in the range of 0.04 to 0.5% by weight.

The solution to be mixed with the specimen may further include an anionic surfactant or zwitterionic surfactant having a steroid skeleton. Use of a combination of the anionic surfactant or zwitterionic surfactant having a steroid skeleton with the zwitterionic surfactant comprising the hydrocarbon chain is effective in treatment of the outer membrane of SARS-CoV-2, thereby enhancing the extraction efficiency of the target protein (for example, N protein). The anionic surfactant or zwitterionic surfactant having the steroid skeleton may be used only one kind, or two or more kinds thereof may be used concurrently.

The anionic surfactant having a steroid skeleton is typically a bile acid or a derivative thereof retaining a steroid skeleton, or a salt thereof. More specific examples of such an anionic surfactant include deoxycholic acid, chenodeoxycholic acid, ursodeoxycholic acid, hyodeoxycholic acid, cholic acid, glycocholic acid, taurocholic acid, hyocholic acid, 5α-cyprinol, lithocholic acid, taurodeoxycholic acid, taurocholic acid, as well as the salts thereof. The salts are the same as those described before.

The zwitterionic surfactant having the steroid skeleton is typically a derivative of a bile acid obtained by derivatizing the bile acid in such a way as to have a cationic portion or a salt thereof having the steroid skeleton. More specifically, illustrative examples of such a zwitterionic surfactant include CHAPS, CHAPSO, and salts thereof. The salts are the same as those described before.

As for the anionic surfactant or zwitterionic surfactant having the steroid skeleton, the anionic surfactant having the steroid skeleton is preferable.

The concentration of the anionic surfactant or zwitterionic surfactant having the steroid skeleton (final concentration in the mixed solution comprising the specimen) is not particularly restricted so far as the concentration thereof is enough for mixing with the specimen. Although the concentration changes in accordance with the factors such as the specimen and the target molecule to be immunologically detected, the concentration may be, for example, in the range of 0.001 to 10% by weight, preferably in the range of 0.01 to 5% by weight, more preferably in the range of 0.05 to 4% by weight, and still more preferably in the range of 0.12 to 2.0 by weight.

The solution to be mixed with the specimen may further include a nonionic surfactant. The surfactant having the steroid skeleton may form a micelle when this is used as the solution with a high concentration, depending on the surfactant. The nonionic surfactant can suppress not only formation of the micelle but also a non-specific reaction in the immunological detection. The nonionic surfactant may be used only one kind, or two or more kinds of them may be used concurrently.

Illustrative examples of the nonionic surfactant include a polyoxyethylene sorbitan fatty acid ester [for example, TWEEN (registered tradename) series (for example, TWEEN 20, TWEEN 40, and TWEEN 80)], polyoxyethylene octyl phenyl ether [for example, TRITON (registered tradename) series (for example, Triton X-100, Triton X-114, Triton X-305, Triton X-405, and Triton X-705)], N-D-gluco-N-methylalkane amide [for example, MEGA series (for example, MEGA 8 and MEGA 10)], and nonionic surfactants having a polyoxyethylene alcohol structure (for example, alcohol ethoxylate and polyoxyethylene-polyoxyalkylene block block polymer).

The concentration of the nonionic surfactant (final concentration in the mixed solution comprising the specimen) is not particularly restricted so far as the concentration thereof is enough for mixing with the specimen. Although the concentration changes in accordance with the factors such as the specimen and the target molecule to be immunologically detected, the concentration may be, for example, in the range of 0.001 to 10% by weight, preferably in the range of 0.01 to 5% by weight, and more preferably in the range of 0.02 to 1% by weight, while preferably in the range of 0.04 to 0.5% by weight.

In the immunological detection, by using one or more antibodies to a target molecule that constitutes SARS-CoV-2, the target molecule can be detected. The one or more antibodies to the target molecule may be mixed in such a way that this is included in the above-mentioned solution. Alternatively, the one or more antibodies to the target molecule may be included in a solution (second solution) that is different from the above-mentioned solution (first solution). In this case, this different solution comprising the one or more antibodies to the target molecule may be mixed with the specimen at the same time with the above-mentioned solution (first solution), or after the specimen and the above-mentioned solution (first solution) are mixed, may be mixed with the mixed solution. By using such an antibody, the target molecule can be detected.

Although the target molecule is not particularly restricted so far as this is the substance that can be retained by SARS-CoV-2 and detectable by an antibody, a protein is preferable. Such a protein is not particularly restricted so far as this is the protein that can be retained by SARS-CoV-2. Illustrative examples thereof include a nucleocapsid protein (N protein), an S protein, an E protein, and an M protein. The protein is preferably the N protein. Accordingly, the one or more antibodies to the target molecule that constitutes SARS-CoV-2 is/are preferably one or more antibodies to the N protein. The SARS-CoV-2 N protein is the same as those described before.

The antibody and the preparation method thereof are the same as those described before.

In one embodiment, the antibody may be a solid phase antibody. The solid phase antibody is the same as those described before.

In another embodiment, the antibody may be a labelled antibody. The labelled antibody is the same as those described before.

According to the present invention, the target molecule may be detected by an immunological method using the antibodies described above. Such an immunological method is the same as those described before.

Detection of the label may be done with the method appropriately chosen in accordance with the label. Details thereof are the same as those described before.

In a specific embodiment, the method of the present invention may be carried out by the sandwich method. The sandwich method is the same as the one described before.

In another specific embodiment, the method of the present invention may be carried out by an immunochromatographic method. The immunochromatographic method is the same as the one described above except that the detection substance is not limited to the N protein, but subjected to all the target molecules.

When the target molecule is the SARS-CoV-2 N protein, preferably, the SARS-CoV-2 N protein may be detected by using the antibodies or combinations thereof as described before.

The present invention also provides the reagent of immunologically detecting SARS-CoV-2, including the following (1) and (2):
(1) a zwitterionic surfactant comprising a hydrocarbon chain with the chain length of 12 to 18 carbon atoms, and
(2) one or more antibodies to a target molecule that constitutes SARS-CoV-2.

The reagent of the present invention may further include the following component(s) (3) and/or (4):
(3) an anionic surfactant or zwitterionic surfactant having a steroid skeleton; and/or
(4) a nonionic surfactant.

Details of the terms of the zwitterionic surfactant comprising the hydrocarbon chain, the one or more antibodies, and the like in the reagent of the present invention (for example, definitions, examples, and preferred examples) are the same as those described in the method of the present invention.

The reagent of the present invention may have the zwitterionic surfactant comprising the hydrocarbon chain and the one or more antibodies in the mixed form (for example, in the mixed solution form stored in the same container), or in the separated form to each other (for example, in the stored form in different containers). In addition, the reagent of the present invention may include, in addition to the zwitterionic surfactant comprising the hydrocarbon chain and the one or more antibodies, the component(s) (3) and/or (4) in the mixed form or in the separated form to each other. The reagent of the present invention may be provided in the form of a kit as well. The reagent of the present invention may also be provided in the form of a device. For example, all the one or more antibodies may be accommodated in the device.

Alternatively, part of the one or more antibodies may be accommodated in the device while the rest of the antibodies is not necessarily accommodated in the device (for example, in the form stored in a different container). In this case, the antibody not accommodated in the device may be charged into the device at the time of detection.

The reagent of the present invention may include the solid phase antibody and/or the labelled antibody as the one or more antibodies. Alternatively, when the reagent does not include the solid phase antibody and/or the labelled antibody, the reagent may include a solid phase and/or a labelling substance. The solid phase and the labelling substance are the same as those described before. When the labelling substance is an enzyme, the reagent of the present invention may include a substrate of the enzyme (for example, the substance that generates a detection signal, or the substrate that is transformed to a product generating a detection signal by the enzyme, or the substrate in the reaction capable of conjugating with other enzyme reaction using the substrate that generates a detection signal or the substrate that is transformed to a product generating a detection signal by the enzyme).

The reagent of the present invention may further include the antibodies or the combinations thereof as described above.

The reagent of the present invention is not particularly restricted so far as this can be used in the immunological measurement method; and thus the reagent can have a composition in accordance with the immunological measurement method. Preferably, the reagent of the present invention may be the reagent that is used in the sandwich method. Therefore, the reagent of the present invention may include a solid phase that is used in the sandwich method (for example, particles such as magnetic particle, a well plate, and a membrane), or a solid phase antibody immobilized to the above-mentioned solid phase. Also, the reagent of the present invention may include a device including a solid phase that is used in the immunochromatographic method (namely, the above-mentioned immunochromatographic cartridge), or a solid phase antibody immobilized to the above-mentioned solid phase. The reagent of the present invention may include a standard of a target molecule (for example, a protein standard such as the N protein standard).

### EXAMPLES

Next, the present invention is more specifically described by Examples, but the present invention is not limited to the following Examples. As far as the quantities in the following Examples are concerned, means by weight (wt %).

### Example 1: Preparation of Antibodies

Antibodies were prepared in accordance with the method described in international Publication No. WO02005/042579. Specifically, the method is as described below.

### (1) Production and Purification of Recombinant N Protein

The gene of the nucleocapsid protein (N protein) of the SARS coronavirus (SARS-CoV) was inserted into an expression plasmid to obtain the plasmid pWS-N. By using this plasmid, E. coli was transformed to obtain an ampicillin-resistant transformed E. coli. The nucleotide sequence and amino acid sequence of the N protein are described in SEQ ID NOs:1 and 2, respectively.

The transformant thereby obtained was cultured at 37° C. in 2 mL of the LB medium containing 50 μg/mL of ampicillin. This transformant was grown by the pre-culturing; then, after the density thereof reached about 0.7 OD at 600 nm, 0.4 mM of IPTG was added to it for expression induction. After 18 hours of culturing, E. coli was recovered by centrifugal separation. After the 20 mM Tris-HCl buffer (pH 8.0) containing 0.1 mM PMSF was added to the recovered E. coli, this was subjected to the ultrasonic disintegration under ice cooling. After centrifugation, ammonium sulfate was added to the soluble fraction to recover the 20 to 40% ammonium sulfate fraction. This ammonium sulfate fraction was applied to SP Sepharose Fast Flow (Amersham plc) that had been equilibrated with a 0.1 M NaCl/8 M urea/20 mM phosphate buffer (pH 6.9); then, this was eluted and purified with a 0.2 M NaCl/8 M urea/20 mM phosphate buffer (pH 6.9). The eluted fraction was dialyzed against a 0.2 M NaCl/20 mM Tris-HCl buffer (pH 8.0) to obtain the purified recombinant N protein (CoV) solution. Only single band was recognized by confirmation of the purification degree of the recombinant N protein with SDS-PAGE and the Western blotting.

### (2) Preparation of Monoclonal Antibody

A mouse was immunized with the obtained recombinant N protein (CoV); then, the splenic lymphocyte of the mouse was fused with the myeloma cell to prepare the anti-N protein monoclonal antibody. Namely, the first immunization was carried out to the BALB/C mouse with 50 to 100 μg/mouse of the recombinant N protein that had been emulsified by the Freund's complete adjuvant; then, after 2 to 3 weeks from it, an additional immunization was carried out with 50 to 100 μg/mouse of the same antigen that had been emulsified with the Freund's incomplete adjuvant. The antibody titer was checked by the solid phase ELISA using the 96-well ELISA plate coated with the recombinant N protein. To the mouse that had been recognized its antibody titer increased, 25 to 100 μg of a free recombinant N protein was intravenously administered; then, after 3 to 4 days from it, a spleen was taken from the mouse to prepare the spleen cells. The mouse myeloma cells (P3U1) that had been cultured previously with the RPMI-1640 culture medium were mixed with the spleen cells at the ratio of 1:2 to 1:5; then, the cell fusion of them were carried out by using PEG. After the fused cell was floated on the HAT culture medium, this was dispensed in the 96-well culture plate, and then cultured in a CO₂ incubator at 37° C.

Screening of the antibody was carried out with the solid phase ELISA described above. Namely, the recombinant N protein with the concentration of 1 μg/mL was dispensed to the 96-well ELISA plate with the amount of 50 μL/well in each well followed by leaving this at 4° C. for one overnight to conduct the adsorption. After the wells were blocked by a 1% skimmed milk, they were washed three times with a washing buffer (PBS containing 0.05% Tween), and then added with 50 μL of the culture supernatant after the cell fusion on the plate to carry out the reaction at 37° C. for 1 hour. After washing with the above-mentioned washing solution three times, the POD-labelled anti-mouse immunoglobulin antibody (DACO A/S) was added to further carry out the reaction at 37° C. for 1 hour. After washed with the washing buffer four times, the substrate ABTS was added; then, the colored wells were selected. Next, the cells in the selected well were transferred to the 24-well culture plate to conduct culturing in the CO₂ incubator at 37° C., which was then followed by limiting dilution to make single clone. In this way, various monoclonal antibodies were obtained.

Similarly, a mouse was immunized with, in place of the recombinant N protein (CoV), the KLH conjugate of the synthetic peptide (N5 peptide: GQTVTKKSAAEASKKPRC: SEQ ID NO:3) as the immunogen, to obtain the anti-N protein monoclonal antibody.

### (3) Confirmation of Reactivity of Monoclonal Antibody

The reactivity of each of the established monoclonal antibodies to the wild type antigen (N protein originated from SARS-CoV) was confirmed with the Western blotting (WB) using the concentrated virus suspension solution as the specimen. The Vero E6 cell was infected with the SARS virus Hanoi strain and cultured in the CO₂ incubator for 48 hours; then, this was centrifuged at 2,000 rpm for 15 minutes to prepare the virus culture supernatant (TCID50: 7.95×10⁶/mL). After this culture supernatant was inactivated at 56° C. for 90 minutes, 31.5 mL thereof was centrifuged at 30,000 rpm for 3 hours using Hitachi ultracentrifuge (40T rotor). To the precipitate thereby obtained, 0.3 mL of the Tris-NaCl-EDTA buffer was added followed by pipetting to obtain the concentrated virus suspension solution. To this suspension solution, the same amount of the specimen treatment solution for electrophoresis was added followed by a heat treatment to obtain the specimen for analysis. After carrying out SDS-PAGE with 12.5% gel, the specimen was transferred onto a nitrocellulose membrane to obtain a transferred membrane for WB (antigen-transferred WB membrane). After the transferred membrane was blocked with the skimmed milk, each monoclonal antibody was shaken with the antigen-transferred WB membrane at room temperature for 1 hour to carry cut the reaction; then, this was washed with the washing buffer three times (washing by shaking for 5 minutes). Next, after the POD-labelled anti-mouse immunoglobulin antibody was added, the reaction was further carried cut at room temperature for 1 hour. After washing with the washing buffer four times (washing by shaking for 5 minutes), a 4-chloronaphthol solution was added as the substrate; then, the band was confirmed. The monoclonal antibody whose band was confirmed at the position corresponding to the N-protein with the molecular weight of a little bit less than 50 kD was selected.

The selected monoclonal antibody was further subjected to the WB using the recombinant N protein of SARS-CoV-2 prepared by the same way as (1) and the common coronavirus N protein; then, the one bound to the recombinant N protein of SARS-CoV-2 but not bound to the N protein of the common coronavirus N protein was considered as the anti-N protein monoclonal antibody. In the Examples described hereinafter, unless otherwise specifically mentioned, the SARS-CoV-2 N protein was used as the N protein.

### Example 2: Identification of Antibody's Reacting Region (Part 1)

With regard to a plurality of the anti-N protein monoclonal antibodies obtained in Example 1, the epitopes thereof were identified by the WB method using the recombinant antigen having a partial sequence of the N protein.

First, to the solution containing recombinant antigens of the whole length of the N protein of SARS-CoV-2 (419 amino acids), NTD (the 44th to 180th amino acids), CTD (the 247th to 364th amino acids), and the C-terminal peptide of the N protein (the 330th to 419th amino acids), these having been prepared in the same way as Example 1, the same amount of the specimen treatment solution for electrophoresis was added, which was followed by a heat treatment to obtain the specimen for analysis; then, SDS-PAGE and WB were carried out in the same way as Example 1 (3) to confirm the reactivity of each of the N protein monoclonal antibodies.

As a result, it was confirmed that 6 antibodies (anti-N protein antibodies N1 to N6) react with the whole length antigen and the NTD antigen and that 6 antibodies (anti-N protein antibodies C1 to C6) react with the whole length antigen and the CTD antigen; thus, a plurality of the antibodies that respectively react to NTD and CTD could be obtained. In addition, the antibodies (anti-N protein antibodies C12 and C13), recognizing the region contained in the 365th to 419th amino acid sequence, that react with the whole length antigen and the C-terminal peptide but do not react with the NTD and CTD antigens were obtained. In addition, a plurality of the anti-N protein monoclonal antibodies (P1 and P2), recognizing the 243rd to 259th amino acid region in the N protein, could be obtained by immunizing the mouse with the KLH conjugate of the synthetic peptide (N5 peptide: GQTVTKKSAAEASKKPRC: SEQ ID NO:3) as the antigen.

### Example 3: Preparation of Solid Phase Antibody (Antibody Solid-Phased Magnetic Particle)

The anti-N protein monoclonal antibody obtained as described above was chemically bonded to magnetic particles (Fujirebio Inc.) by using the carboxyl-amine crosslinking agent (carbodiimide; Thermo-Fisher Scientific) in accordance with the product manual thereof to obtain the solid phase antibody (antibody solid-phased magnetic particles).

### Example 4: Preparation of Labelled Antibody (Alkaline phosphatase-labelled Anti-N Protein Monoclonal Antibody)

The anti-N protein monoclonal antibody obtained as described above was mixed with pepsin in the 0.1 M citrate buffer (pH 3.5); then, the resulting mixture was allowed to statically leave at 37° C. for 1 hour to carry out the digestion by pepsin. After termination of the reaction, gel-filtration purification thereof was carried out to obtain an antibody having the Fc region removed. Next, this was thiolated with 2-mercaptoethyl ammonium chloride (2-MEA), which was then followed by desalting treatment to obtain a Fab′ fragment.

The alkaline phosphatase treated with N-Succinimidyl 4-Maleimidobutyrate (GMBS) and the Fab′ fragment prepared from each of the anti-N protein monoclonal antibodies were mixed with the mole ratio of 1:1 to 3:1 to carry out the coupling reaction. To this coupling solution, 2-mercaptoethyl amine hydrochloride and acetamide iodide were added to terminate the reaction. The peak corresponding to the molecular weight of Fab′ and ALP with the ratio of 1:1 to 3:1 in gel filtration was pooled to obtain the labelled antibody (ALP-labelled anti-N protein monoclonal antibody).

### Example 5: Sandwich Immunoassay Using Particles

To 50 μL of the particle suspension dilution solution (50 mM of Tris, 1% of BSA, 150 mM of NaCl, 1 mM of EDTA, and 0.1% of NaN₃) containing 0.03% of the antibody solid-phased magnetic particles prepared in Example 3, 100 μL of the solution containing the recombinant N protein or the specimen was added to react at 37° C. for 8 minutes.

After the reaction, the B/F separation was carried out by a magnet, which was then followed by washing with the Lumipulse (registered trademark) washing solution (Fujirebio Inc.); then, the reaction was carried out at 37° C. for 8 minutes with addition of 50 μL of the labelled antibody fluid (50 mM of MES, 150 mM of NaCl, 1% of BSA, 3 mM of MgCl₂, and 0.3 mM pf ZnCl₂) containing 1 μg/mL of the ALP-labelled antibody prepared in Example 4. The B/F separation was carried out by a magnet; then, after washing with the Lumipulse washing solution, 200 μL of the Lumipulse (registered tradename) substrate solution (Fuji Rebio Inc.) containing 3-(2′-spiroadamantane)-4-methoxy-4-(3″-phosphoryloxy) phenyl-1,2-dioxetane disodium salt (AMPPD) was added to carry out the enzymatic reaction at 37° C. for 4 minutes. Then, the luminescence amount was measured at the wavelength of 463 nm.

### Example 6: Immunochromatographic Method

As illustrated in FIG. 1, in the matrix formed of the nitrocellulose membrane with the width of 3.7 mm and the length of 50 mm, at the position 15 mm from the end in the side of the developing solution absorbing zone, 0.5 μL of the aqueous solution containing the anti-N protein monoclonal antibody was spotted on the nitrocellulose membrane and dried to form the detecting zone. At the position 12 mm from the end in the side of the developing solution absorbing zone, the anti-alkaline phosphatase (ALP) antibody solution (sodium carbonate buffer containing 0.15 M of NaCl) was spotted and dried to form the development confirming part. Also, 0.9 μL of the 5-bromo-4-chloro-3-indolyl phosphate disodium (BCIP) solution as the substrate was spotted in a form of a line to form the substrate zone. Next, 3 μL of the solution containing the ALP-labelled anti-N protein monoclonal antibody (labelled antibody) was spotted on the matrix and then dried to form the labelled reagent zone formed of the enzyme-labeled reagent pad.

The matrix, the developing solution pad, the enzyme-labelled reagent pad, and the absorbing pad (highly water-retentive filter paper) were piled as illustrated in FIG. 1; then, they were fixed by an adhesive tape. This was further fixed to a plastic case having the developing solution tank 11 to obtain the immunochromatographic cartridge for detection of the SARS-N protein.

Next, 20 μL of the solution containing the recombinant N protein or of specimen was mixed with 100 μL of the treatment solution; then, the mixture was allowed to statically leave at room temperature for 5 minutes. The N protein in the mixed solution thereby obtained was detected by using the obtained immunochromatographic cartridge. Specifically, after 20 μL of the mixed solution was dropped on the labelled antibody zone in the immunochromatographic cartridge, the developing solution tank was broken; then, the developing solution was fed to the developing solution pad and the matrix upper end to start the measurement. After 30 minutes from the start of the measurement, development of the developing solution was confirmed by coloring at the development confirming part 10; then, the color intensity in the detecting zone was visually measured.

### Example 7: Identification of Antibody's Reacting Region (Part 2)

In accordance with the sandwich immunoassay method in Example 5, the recombinant N protein (SARS-CoV-2 or SARS-CoV) was measured to classify into groups corresponding to the reaction region of each N protein monoclonal antibody. Specifically, the recombinant N protein was measured by using, as the antibody solid-phased magnetic particles (solid phase antibody) and the ALP-labelled antibody, N1 to N6 antibodies, C1 to C6 antibodies, and C12 antibody.

As a result, when the recombinant N protein (0 and 10 ng/mL) was measured by using, as the solid phase antibody and the labelled antibody, the N1 antibody and the N2 antibody, the luminescence amount ratio at 10 ng/mL to the luminescence amount at 0 ng/mL (background value) was very small, so that it was confirmed that they react in the overlapping region (Table 1). Similarly, when the recombinant N protein (0 and 1 ng/mL) was measured by using, as the solid phase antibody and the labelled antibody, the N4, N5, and N6 antibodies (N6 antibody group), the luminescence amount difference obtained by subtracting the luminescence amount at 0 ng/mL from the luminescence amount at 1 ng/mL was very small, so that it was confirmed that these antibodies recognize the overlapping region (Table 2). On the other hand, when the N1 antibody and N3 antibody were used as the solid phase antibody and the labelled antibody, the luminescence amount difference was smaller as compared with the combinations of other antibodies, so that it was confirmed that they are partially competitive. Because the N1 antibody has higher reactivity with other NTD antibodies than the N3 antibody in the sandwich immunoassay, it is thought that the N1 antibody group (N1 and N2 antibodies) is more usable than the N3 antibody in the sandwich immunoassay that uses a plurality of the antibodies.

TABLE 1
Reactivity to SARS-Cov-2 N protein (1)

| Luminescence amount | | Labelled antibody | | | |
|---|---|---|---|---|---|
| ratio (10 ng/mL/0 ng/mL) | | N1 | N2 | N4 | N5 |
| Solid phase | N1 | 1.6 | 8.3 | 4,471.7 | 2,433.6 |
| antibody | N2 | 2.0 | 7.8 | 4,424.1 | 2,632.1 |
| | N4 | 968.0 | 384.9 | 72.9 | 9.3 |
| | N5 | 1,186.5 | 860.1 | 54.7 | 6.8 |

TABLE 2
Reactivity to SARS-CoV-2 N protein (2)

| Luminescence amount | | | | | | |
|---|---|---|---|---|---|---|
| difference | | Labelled antibody | | | | |
| (1 ng/mL − 0 ng/mL) | | N1 | N3 | N4 | N5 | N6 |
| Solid phase | N1 | 44 | 2,407 | 21,943 | 11,677 | 41,176 |
| antibody | N3 | 159 | 2 | 4,393 | 2,122 | 5,213 |
| | N4 | 3,621 | 13,314 | 105 | −4 | 13 |
| | N5 | 4,713 | 11,9513 | 76 | −8 | −7 |
| | N6 | 8,831 | 10,841 | 132 | 39 | 13 |

Similarly, C1 to C6 antibodies were studied. It was confirmed that the C1 antibody and the C2 antibody recognize the overlapping region, that the C3 antibody and the C4 antibody recognize the overlapping region, and that the C5 antibody and the C6 antibody recognize the overlapping region (Tables 3 to 5). However, the C5 antibody and the C6 antibody showed low luminescent amounts in any combination, so that it was confirmed that the antibodies to the region that the C5 antibody group (C5 and C6 antibodies) recognizes have low reactivities in the sandwich immunoassay measurement.

TABLE 3
Reactivity to SARS-CoV N protein (1)

| Luminescence amount | | Labelled antibody | | |
|---|---|---|---|---|
| difference (10 ng/mL-0 ng/mL) | | C1 | C3 | C12 |
| Solid phase | C1 | 19,980 | 1,079,590 | 1,025,492 |
| antibody | C2 | 2,982 | 244,857 | 805,214 |

TABLE 4
Reactivity to SARS-CoV-2 N protein (3)

| Luminescence amount | | Labelled antibody | | |
|---|---|---|---|---|
| difference (1 ng/mL-0 ng/mL | | C2 | C3 | C12 |
| Solid phase | C2 | 63 | 20,495 | 56,541 |
| antibody | C3 | 28,704 | −148 | 58,987 |
| | C4 | 26,237 | 78 | 46,620 |
| | C12 | 31,267 | 72,145 | 5 |

TABLE 5
Reactivity to SARS-CoV-2 N protein (4)

| Luminescence amount | | Labelled antibody | | | |
|---|---|---|---|---|---|
| ratio (10 ng/mL/0 ng/mL) | | C3 | C5 | C6 | C12 |
| Solid phase | C3 | 2.9 | 7.3 | 12.3 | 549.2 |
| antibody | C4 | 23.9 | 2.2 | 3.8 | 1,107.2 |
| | C5 | 403.6 | 3.1 | 5.0 | 273.8 |
| | C6 | 576.4 | 3.1 | 7.5 | 404.5 |
| | C12 | 847.1 | 22.8 | 52.8 | 1.3 |

### Example 8: Study about Combination of Antibodies (Part 1)

Combination of the solid phase antibody and the labelled antibody in the antibodies grouped in Example 7 and the C12 antibody was studied. In a similar way to the sandwich immunoassay method in Example 5, the recombinant N protein was measured using the combinations of the antibodies as listed in Table 6. Measurements each were done with N=2.

The results are listed in Table 6. In Table 6, the ratio of the average value of the luminescence amounts from the 1 ng/mL specimen to the average value of the luminescence amounts from the 0 ng/mL specimen is listed (S/N ratio).

TABLE 6
Reactivity to SARS-CoV-2 N protein (5)

| Luminescence amount | | Labelled antibody | | | |
|---|---|---|---|---|---|
| ratio (1 ng/mL/0 ng/mL) | | N1 | N4 | C3 | C12 |
| Solid phase | N1 | 1 | 847 | 1201 | 828 |
| antibody | N4 | 165 | 9 | 866 | 629 |
| | C3 | 988 | 1,064 | 3 | 871 |
| | C12 | 842 | 881 | 895 | 1 |

As a result, it was confirmed that the combinations between the N1 antibody and the C3 antibody, between the N1 antibody and the C12 antibody, between the N4 antibody and the C3 antibody, between the N4 antibody and the C12 antibody, and between the C3 antibody and the C12 antibody are preferable, because these combinations exhibit high S/N ratio in both combinations in the combination of the solid phase antibody and the labelled antibody in the sandwich immunoassay.

### Example 9: Study about Combination of Antibodies (Part 2)

Combination of the solid phase antibody and the labelled antibody in the antibodies grouped in Example 7, the P1 antibody, and the C12 antibody was studied. By the immunochromatographic method in Example 6, the specimen containing 10 ng/mL of the recombinant N protein was measured using the combinations of the antibodies as listed in Tables 7 and 8. In addition, by using the combinations of the antibodies as listed in Tables 7 and 8, development of the developing solution was confirmed after 30 minutes (Table 7) and 60 minutes (Table 8) from start of the measurement by the color intensity in the development confirming part 10, and then, the color intensity in the detecting zone was measured visually. The results are listed in Tables 7 and 8. In Tables 7 and 8, the color intensities in the detecting zone under different conditions are listed. As the number is more, the color is deeper; “w” indicates weaker. Namely, the color intensity is in the order of 4.5>4>3>2>1>w (from strong to weak). The blank cells indicate there was no measurement.

TABLE 7
Reactivity to SARS-CoV-2 N protein (6)

| | | Labelled antibody | | | | | | | |
|---|---|---|---|---|---|---|---|---|---|
| | | N1 | N2 | N4 | N5 | C3 | C5 | C6 | C12 |
| Solid phase | N1 | | | 2 | 3 | 3 | w | 1 | 3 |
| antibody | N2 | | | 2 | 3 | 3 | w | 1 | 3 |
| | N4 | 3 | 3 | | | 3 | w | 1 | 3 |
| | C12 | 4 | 3 | 3 | 3 | 4.5 | 3 | 3 | |

TABLE 8
Reactivity to SARS-CoV-2 N protein (7)

| | | Labelled antibody | | |
|---|---|---|---|---|
| | | N1 | C3 | N4 |
| Solid phase | C1 | 1 | 2 | 1 |
| antibody | P1 | 2 | 4 | 2 |
| | N6 | 2 | 4 | |

As a result, similarly to the sandwich immunoassay that uses the particles, strong color intensity was confirmed in the combinations between the N1 antibody/the N2 antibody and the C3 antibody, between the N1 antibody and the C12 antibody, between the N4 antibody and the C3 antibody, between the N4 antibody and the C12 antibody, and between the C3 antibody and the C12 antibody. In the combination between the N1 antibody/the N2 antibody and the N4 antibody, color intensity was relatively high.

The combinations of the C1 antibody and the P1 antibody with other antibodies were also evaluated. It was found that the C1 antibody exhibited relatively strong color intensity when combined with the C3 antibody. On the other hand, the P1 antibody exhibited strong color intensity when combined with the C3 antibody and relatively strong color intensity when combined with the N1 antibody and with the N4 antibody.

### Example 10: Study about Combination of Antibodies (Part 3)

In Example 8 and in Example 9, the combination of a single solid phase antibody with a single labelled antibody was studied. Even such a combination could construct the measurement system with a sufficient sensitivity; but it is more preferable to use the combination of the solid phase antibodies that recognize different regions and/or the labelled antibodies that recognize different regions. Therefore, the immunoassay system was constructed by using, as the labelled antibody, one of the antibodies that were recognized effective when used singly in Examples 8 and 9, and, as the solid phase antibody, a plurality of other antibodies; then, the specimen containing 10 ng/mL of the recombinant N protein (SARS-CoV) was evaluated in the same way as Example 8. The solid phase antibody was prepared by chemically bonding a plurality of the antibodies to the particles at the same time in accordance with the preparation method in Example 3. The results are listed in Table 9.

As a result, when the mixture of the C3 antibody and the C12 antibody was used, significant increase in the luminescence amount (count at 1,000 pg/mL) and the S/N ratio were recognized. In particular, in the case where the N1 antibody or the N4 antibody was used as the labelled antibody, when the mixture of the C3 antibody and the C12 antibody was used as the solid phase antibody, it was confirmed that the luminescence amount and the S/N ratio increased as compared when the C3 antibody and the C12 antibody were used singly as the solid phase antibody. On the other hand, in the case where the C3 antibody or the C12 antibody was used as the labelled antibody, even when the mixture of the N1 antibody and the N4 antibody was used as the solid phase antibody, both the count value and the S/N ratio did not increase. Accordingly, it was confirmed that the combination of the C3 antibody and the C12 antibody is effective in enhancement of the sensitivity in the sandwich immunoassay, not because of mere use of a plurality of the antibodies as the solid phase antibody.

TABLE 9
Reactivity to SARS-CoV N protein (2)

| Labelled | Solid phase | | Average count | | S/N |
|---|---|---|---|---|---|
| antibody | antibody | | 0 pg/mL | 1,000 pg/mL | 1,000/0 |
| N1 | N1 | | 211 | 216 | 1 |
| | C12 | | 254 | 213,764 | 842 |
| | N4 | | 308 | 50,851 | 165 |
| | C3 | | 202 | 199,517 | 988 |
| | C12 | N4 | 245 | 175,116 | 716 |
| | C12 | C3 | 229 | 225,992 | 989 |
| | N4 | C3 | 226 | 75,019 | 332 |
| C12 | N1 | | 225 | 185,994 | 828 |
| | C12 | | 548 | 665 | 1 |
| | N4 | | 327 | 205,245 | 629 |
| | C3 | | 234 | 203,348 | 871 |
| | N1 | C12 | 425 | 1,585 | 4 |
| | N1 | N4 | 255 | 168,693 | 662 |
| | N1 | C3 | 221 | 199,922 | 905 |
| N4 | N1 | | 198 | 167,312 | 847 |
| | C12 | | 269 | 236,610 | 881 |
| | N4 | | 300 | 2,699 | 9 |
| | C3 | | 212 | 225,033 | 1,064 |
| | N1 | C12 | 248 | 187,426 | 756 |
| | N1 | C3 | 216 | 180,804 | 837 |
| | C12 | C3 | 227 | 266,502 | 1,177 |
| C3 | N1 | | 227 | 271,927 | 1,201 |
| | C12 | | 268 | 239,924 | 895 |
| | N4 | | 315 | 272,825 | 866 |
| | C3 | | 251 | 630 | 3 |
| | N1 | C12 | 253 | 300,127 | 1,186 |
| | N1 | N4 | 240 | 234,377 | 977 |
| | C12 | N4 | 252 | 232,798 | 924 |

### Example 11: Identification of Antibody's Reaction Region (Part 3)

With regard to the antibodies whose effectiveness was confirmed in Examples 8 to 10, the epitopes in the N protein (SARS-CoV-2) were analyzed.

With regard to the N1 antibody group (N1 and N2 antibodies) and the N6 antibody group (N4 to N6 antibodies), which recognize NTD, the reaction regions were analyzed by WB that uses the recombinant antigen composed of the NTD's N-terminal region (the 44th to 112nd amino acids), the NTD's central region (the 79th to 147th amino acids), and the NTD's C-terminal region (the 113rd to 180th amino acids), and the recombinant antigen (trNP1, trNP2, trNP3, and trNP4 antigens) composed of the 1st to 119th, the 110th to 229th, the 220th to 339th, and the 330th to 419th amino acid regions in the N protein; these had been prepared in the same way as Example 1. As a result, it was found that the N1 antibody group recognizes the 44th to 78th amino acid region in the N protein, and that the N6 antibody group recognizes the 120th to 147th amino acid region in the N protein.

Similarly, with regard to the C3 antibody group and the C1 antibody group, which recognize CTD, the reaction regions were analyzed by WB, the sandwich assay, as well as based on the reactivities with synthetic peptides and with other antibodies, using the recombinant antigen composed of the CTD's N-terminal region (the 247th to 305th amino acids), the CTD's central region (the 276th to 334th amino acids), and the CTD's C-terminal region (the 306th to 364th amino acids), as well as the trNP1, trNP2, trNP4, and trNP4 antigens; these had been prepared in the same way as Example 1. As a result, it was found that the C3 antibody group recognizes the 260th to 305th amino acid region in the N protein, and that the C1 antibody group recognizes the 306th to 339th amino acid region in the N protein.

The antibody's epitopes obtained by the analysis are summarized as follows.

TABLE A
Relationship between Antibody and Epitope

| Antibody | Epitope in SARS-CoV-2 |
|---|---|
| N1 | The 44th to 78th amino acid region |
| N6 | The 120th to 147th amino acid region |
| P1 | The 243rd to 259th amino acid region |
| C1 | The 306th to 339th amino acid region |
| C3 | The 260th to 305th amino acid region |
| C12 | The 365th to 419th amino acid region |

### Example 12: Study about Combination of Antibodies (Part 4)

In Example 10, in the case where the N1 antibody or the N4 antibody was used, it was found that the mixture of the C3 antibody and the C12 antibody is useful as the solid phase antibody. In order to confirm that the combination of these antibodies is useful also for SARS-CoV-2, the solution containing 100 pg/mL of the recombinant N protein was measured in the same way as Example 5 by using the combinations of the solid phase antibodies and the labelled antibodies as listed in Table 10.

Specifically, 100 μL of the solution containing the recombinant N protein and 20 μL of the treatment solution (Tris buffer, 150 mM of NaCl, 0.1% of NaN₃, and 2.5% of C16APS) were added to 50 μL of the particle suspension dilution solution containing the solid phase antibody to carry out the reaction at 37° C. for 8 minutes. After the reaction, the B/F separation was carried out by a magnet, which was then followed by washing with the Lumipulse washing solution; then, 50 μL of the labelled antibody fluid containing the ALP-labelled antibody was added to carry out the reaction at 37° C. for 8 minutes. The B/F separation was carried out by a magnet; then, after washing with the Lumipulse washing solution, 200 μL of the Lumipulse substrate solution containing AMPPD was added to carry out the enzymatic reaction at 37° C. for 4 minutes. The luminescence amount at the wavelength of 463 nm was measured, so that the N protein in each sample was measured. The solid phase antibody was prepared in accordance with Example 3 in such a way that the respective antibodies were chemically bonded to the particles followed by mixing them at 1:1. The labelled antibody was prepared as the ALP-labelled antibody in accordance with Example 4; namely, the GNBS-treated ALP and the Fab′ fragments prepared from a plurality of the anti-N protein monoclonal antibodies were simultaneously mixed such that the mole ratio of each Fab′ fragment to ALP was 1:1 to carry out the coupling reaction.

The results are listed in Table 10. As a result, it was found that when the mixture of the C3 antibody and the C12 antibody was used, the luminescence amount (count at 100 pg/mL) and the S/N ratio were excellently increased. It was confirmed that the use of the combination of the C3 antibody and the C12 antibody was also effective to enhance the sensitivity in the sandwich immunoassay of SARS-CoV-2.

TABLE 10
Reactivity to SARS-COV-2 N protein (8)

| Labelled | | Solid phase | | Average count | | S/N |
|---|---|---|---|---|---|---|
| antibody | | antibody | | 0 pg/mL | 100 pg/mL | 100/0 |
| N6 | C12 | N1 | P1 | 1,801 | 57,261 | 31.8 |
| P1 | N6 | N1 | C12 | 1,477 | 28,781 | 19.5 |
| N1 | P1 | N6 | C12 | 1,360 | 41,101 | 30.2 |
| N1 | N6 | P1 | C12 | 1,144 | 28,329 | 24.8 |
| N1 | N6 | C3 | C12 | 1,305 | 205,862 | 157.7 |

### Example 13: Study about Combination of Antibodies (Part 5)

In Example 10 and Example 12, in the case where the N1 antibody or the N4 antibody was used, it was found that use of the mixture of the C3 antibody and the C12 antibody as the solid phase antibody is useful. As described before, there is a possibility of forming a mutant or the like in SARS-CoV-2; and thus it is more preferable to use a mixture of the antibodies recognizing different regions. Accordingly, the evaluation was done about the combination with the P1/P2 antibodies, which belong to the group of the antibodies having different reactivities from the groups of the N1/N2 antibodies, the N4/N6 antibodies, the C3/C4 antibodies, and the C12/C13 antibodies, and which have high reactivities to the N1 antibody, the N4 antibody, and the C3 antibody in Example 10. The combination with the C1/C2 antibodies was also evaluated.

Specifically, the combinations of the solid phase antibodies and the labelled antibodies listed in Tables 11-1 to 11-3 were evaluated in the same way as Example 8. The solid phase antibodies were prepared in accordance with example 3 by chemically bonding a plurality of the antibodies to the particles at the same time. The labelled antibodies were prepared in accordance with Example 4. Namely, the GMBS-treated alkaline phosphatase (ALP) and the Fab′ fragments prepared from a plurality of the anti-N protein monoclonal antibodies were simultaneously mixed such that the mole ratio of each Fab′ fragment to ALP was 1:1; then, the coupling reaction was carried out to obtain the ALP-labelled antibody.

The specimen was prepared by adding the recombinant N protein or the nasal swabs 1 to 5 of the SARS-CoV-2 positive patient (purchased specimen: Boca Biolistics, LLC) to the SARS-CoV-2 negative salvia specimen; then, 100 μL of this specimen was mixed with 170 μL of the specimen treatment solution (Tris buffer, 150 mM of NaCl, 1% of BSA, 0.1% of NaN₃, and 0.25% of C16APS) to obtain the specimen for use.

The results are listed in Tables 11-1 to 11-3. Tables 11-1 to 11-3 list the luminescence amount ratio (S/N ratio) of the luminescence amount measured with 10 ng/mL of the recombinant N protein to the luminescence amount measured with 0 ng/mL of the recombinant N protein, as well as the luminescence amount ratio of each SARS-CoV-2 positive specimen to the luminescence amount measured with 0 ng/mL of the recombinant N protein.

TABLE 11-1
Reactivity to SARS-CoV-2 N protein (9)

| | | | | Combination of labelled antibody | | | | | |
|---|---|---|---|---|---|---|---|---|---|
| | | | | N1, C3, and C12 | | | | | |
| | | | | | Specimen No. | | | | |
| | | | | S/N | 1 | 2 | 3 | 4 | 5 |
| Combination | N6 | P1 | — | 57.6 | 5.50 | 8.35 | 8.17 | 4.56 | 20.62 |
| of solid | P1 | C3 | C12 | 15.9 | 1.22 | 1.56 | 0.79 | 2.13 | 3.31 |
| phase | N6 | P1 | C12 | 21.4 | 1.36 | 4.27 | 2.14 | 3.04 | 9.26 |
| antibody | N6 | P1 | C3 | 58.5 | 4.93 | 13.63 | 4.05 | 4.01 | 14.14 |
| | N6 | P1 | C1 | 53.7 | 5.34 | 9.74 | 4.50 | 5.57 | 15.83 |

TABLE 11-2
Reactivity to SARS-CoV-2 N protein (10)

| | | | | Combination of labelled antibody | | | | | |
|---|---|---|---|---|---|---|---|---|---|
| | | | | N1 and N6 | | | | | |
| | | | | | Specimen No. | | | | |
| | | | | S/N | 1 | 2 | 3 | 4 | 5 |
| Combination | N6 | P1 | — | | | | | | |
| of solid | P1 | C3 | C12 | 59 | 4.50 | 6.83 | 3.64 | 3.25 | 11.64 |
| phase | N6 | P1 | C12 | 5.6 | 2.46 | 3.44 | 3.82 | 2.03 | 11.12 |
| antibody | N6 | P1 | C3 | 6 | 1.26 | 3.64 | 2.81 | 1.66 | 10.00 |
| | N6 | P1 | C1 | 5.5 | 0.80 | 3.57 | 2.63 | 1.07 | 8.65 |

TABLE 11-3
Reactivity to SARS-CoV-2 N protein (11)

| | | | | Combination of labelled antibody | | | | | |
|---|---|---|---|---|---|---|---|---|---|
| | | | | N1, N6, and C1 | | | | | |
| | | | | | Specimen No. | | | | |
| | | | | S/N | 1 | 2 | 3 | 4 | 5 |
| Combination | N6 | P1 | — | 10.6 | 0.80 | 3.86 | 2.05 | 2.63 | 9.05 |
| of solid | P1 | C3 | C12 | 47.5 | 4.24 | 6.82 | 3.50 | 5.02 | 12.72 |
| phase | N6 | P1 | C12 | 13.1 | 0.88 | 4.46 | 2.03 | 2.81 | 0.61 |
| antibody | N6 | P1 | C3 | 14.2 | 1.73 | 4.68 | 2.24 | 7.53 | 8.55 |
| | N6 | P1 | C1 | | | | | | |

As a result, when the C3 antibody and the C12 antibody were used as one of the solid phase antibody and the labelled antibody, and the N6 antibody was used as the other, SARS-CoV-2 in the specimen could be measured with a very high sensitivity. Namely, it was confirmed that when the antibody that recognizes the 260th to 305th amino acid region and the antibody that recognizes the 365th to 419th amino acid region are used as one of the solid phase antibody and the labelled antibody, and the antibody that recognizes the 120th to 147th amino acid region was used as the other, SARS-CoV-2 in the specimen can be measured with a very high sensitivity.

In addition, when the N1 antibody and the P1 antibody were combined, SARS-CoV-2 in the specimen could be measured with a far higher sensitivity. Namely, in addition to the above-mentioned combination, when the antibody that recognizes the 44th to 78th amino acid region and the antibody that recognizes the 243rd to 259th amino acid region were combined, it was confirmed that SARS-CoV-2 in the specimen can be measured with a far higher sensitivity.

Also, even when the C1 antibody is further added to these combinations of the antibodies, SARS-CoV-2 in the specimen could be measured with a sufficient sensitivity. Namely, it was confirmed that the combination further added with the antibody that recognizes the CTD region different from other antibodies is also effective.

### Example 14: Study about Surfactant Used in Reaction Solution

The sample was prepared by adding the nasal swabof the SARS-CoV-2 positive patient (Boca Biolistics, LLC) to the SARS-CoV-2 negative salvia specimen. Then, 100 μL of this sample and 20 μL of the treatment solution containing the surfactant listed in Table 12 (Tris buffer, 150 mM of NaCl, 1% of BSA, 0.1% of NaN₃, 2.5% of surfactant concentration in the treatment solution, and 0.19% as the concentration after mixing of the specimen with the particle suspension solution) were added to 150 μL of the particle suspension dilution solution containing the solid phase antibody to carry cut the reaction at 37° C. for 8 minutes. After the reaction, the B/F separation was carried out by a magnet, which was then followed by washing with the Lumipulse washing solution (Fujirebio Inc.); then, 150 μL of the labelled body fluid containing the ALP (alkaline phosphatase)-labelled antibody prepared in Example 4 was added to carry out the reaction at 37° C. for 8 minutes. The B/F separation was carried out by a magnet; then, after washing with the Lumipulse washing solution, 200 μL of the Lumipulse substrate solution containing AMPPD (3-(2′-spiroadamantan)-4-methoxy-4-(3″-phosphoryloxy) phenyl-1,2-dioxetane disodium salt) was added to carry out the enzymatic reaction at 37° C. for 4 minutes. By measuring the luminescence amount at the wavelength of 463 nm, the N protein in each sample was measured. Here, the N6 antibody and the P1 antibody were used as the solid phase antibody, and the N1 antibody, the C3 antibody, and the C12 antibody were used as the labelled antibody.

The results are listed in Table 12. In Table 12, not only the measured luminescence values but also the rates (%) of the luminescence value under the condition added with the surfactant to the luminescence value under the condition not added with the surfactant are listed.

TABLE 12
Surfactant concentration

| | | | Positive | Positive |
|---|---|---|---|---|
| | | | specimen | specimen |
| | | | (luminescence | (luminescence |
| Condition No. | | Surfactant | value) | rate) |
| No. 1 | Control | — | 2,457 | 100% |
| No. 2 | Anionic | SDS | 963 | 39% |
| No. 3 | | NLS | 1,096 | 45% |
| No. 4 | Nonionic | NP PE-68 | 2,543 | 104% |
| No. 5 | | Tween 20 | 3,323 | 135% |
| No. 6 | | TERGITOL™ | 6,315 | 257% |
| | | 15-S-7 | | |
| No. 7 | | NP40 | 6,715 | 273% |
| No. 8 | Zwitterionic | C12APS | 6,719 | 274% |
| No. 9 | | C12APS | 8,193 | 334% |
| No. 10 | | Arginine | 2,456 | 100% |

SDS: Sodium dodecyl sulfate
NLS: Sodium N-lauroyl sarcosinate
NP PE-68: EWPOL PE-68 (Polyoxyethylene Polyoxypropylene block polymer)
TERGITOL™ 15-S-7: Secondary ethoxylated alcohol
NP-40: Nonyl phenoxypolyethoxylethanol

As a result, when the zwitterionic surfactants having the hydrocarbon chain as the hydrophobic portion (C12APS and C14APS) were used, a very high luminescence value was obtained in the positive specimen, so that the N protein could be detected with high sensitivity. Accordingly, it was found that the zwitterionic surfactant comprising the hydrocarbon chain as the hydrophobic portion is useful in measurement of the SARS-CoV-2 N protein contained in the patient specimen.

Example 15: Study about Surfactant Used in Specimen Treatment Solution (Part 1)

In Example 14, in the measurement of the SARS-CoV-2 N protein, it was found that mixing of the zwitterionic surfactant comprising the hydrocarbon chain as the hydrophobic portion with the specimen is useful. Accordingly, by using the specimen that is treated with the zwitterionic surfactant comprising the hydrocarbon chain as the hydrophobic portion, the N protein was detected by the immunochromatography described in Example 6.

Here, the purchased nasopharyngeal specimen (negative specimen and positive specimen; Boca Biolistics, LLC) was used as the specimen. The Tris buffer (150 mM of NaC1, 1% of BSA, and 0.1% of NaN₃) containing each surfactant was used as the specimen treatment solution. The P1 antibody and the N6 antibody were used as the solid phase antibodies, and the N1 antibody, the C3 antibody, and the C12 antibody were used as the labelled antibodies. In Table 13, the color intensities in the detecting zone under different conditions are listed. As the number is more, the color is deeper; “w” indicates weaker. Namely, color intensity is weaker in the order of 1>1w>1ww (from strong to weak). As a result, it was confirmed that the N protein can be detected with a sufficient sensitivity, as can be listed in Table 13.

TABLE 13
Detection of N protein by
immunochromatographic method
Composition of specimen treatment solution

| | | | | |
|---|---|---|---|---|
| C16APS | 1.00% | 0.50% | 0.25% | |
| C14APS | | | | 0.50% |
| Color intensity | 1w | 1 | 1 | 1ww |

Example 16: Study about Surfactant in Specimen Treatment Solution (Part 2)

In measurement of the SARS-CoV-2 N protein, it was found in Examples 14 and 15 that treatment of the specimen by the zwitterionic surfactant comprising the hydrocarbon chain as the hydrophobic portion is useful.

Next, the zwitterionic surfactant comprising the hydrocarbon chain as the hydrophobic portion and the combination thereof with other surfactant were studied by the sandwich immunoassay method using the particles.

The specimen was prepared by adding 6 μL of the SARS-CoV-2 positive nasopharyngeal specimen (Boca Biolistics, LLC) into 3 mL of the virus transporting solution SGVTM-3R (Sugiyama-gen Co., Ltd); then, this was used. Out of the purchased SARS-CoV-2 positive specimens (Boca Biolistics, LLC), the specimens whose virus amounts were found to be different by the RT-PCR method were chosen as the low-value specimen and the middle-value specimen; then, they were used.

20 μL of the specimen treatment solution (Tris buffer, 150 mM of NaCl, 1% of BSA, and 0.1% of NaN₃) containing the surfactant listed in Table 14 and 0.25% of C16APS were added to 100 μL of the specimen; then, they were incubated at 37° C. for 6.5 minutes. Then, 50 μL of the particle suspension dilution solution containing the solid phase antibody was added to the treated specimen to carry out the reaction at 37° C. for 8 minutes. After the reaction, the B/F separation was carried out by a magnet, which was then followed by washing with the Lumipulse washing solution (Fujirebio Inc.); then, 50 μL of the labelled antibody fluid containing the ALP-labelled antibody prepared in Example 4 was added to carry out the reaction at 37° C. for 8 minutes. The B/F separation was carried out by a magnet; then, after washing with the Lumipulse washing solution, 200 μL of the Lumipuise substrate solution containing AMPPD was added to carry out the enzymatic reaction at 37° C. for 4 minutes. The luminescence amount at the wavelength of 463 nm was measured, so that the N protein in each sample was measured. The standard solutions containing the recombinant N protein with known concentrations (0, 100, 5,000, and 10,000 pg/mL) were similarly measured to prepare the standard curve. From the luminescence amount of each sample and the standard curve, the concentration of the N protein in each sample was obtained. These results are listed in Table 14.

As a result, when deoxycholic acid or cholic acid was further added to the treatment solution, the increase in the luminescence amount was recognized in both the low value specimen (LS) and the middle value specimen (MS). In particular, the additional addition of deoxycholic acid significantly increased the luminescence amount in all the concentrations of the added deoxycholic acid.

Addition of the component such as deoxycholic acid causes gelation of the solution. Even the gelled solution can be used in treatment of the specimen; but when a nonionic surfactant such as Tween 80, Tween 20, or NP-40 was further added to the treatment solution so as to be more readily handled, it was found that gelation of the solution could be prevented, and that a high luminescence amount could be obtained.

TABLE 14
Study about combination of surfactants

| | | | | | | Measured value | | | Ratio to | |
|---|---|---|---|---|---|---|---|---|---|---|
| | | Concentration | Count | | | (pg/mL) | | | condition 1 | |
| Condition | | (%) | NS | LS | MS | NS | LS | MS | LS | MS |
| 1 | No addition | — | 1,208 | 85,361 | 930,587 | 0.02 | 38.32 | 423.09 | 100.0% | 100.0% |
| 2 | CA | 0.50 | 1,233 | 93,373 | 989,464 | 0.03 | 41.96 | 449.91 | 109.5% | 106.3% |
| 3 | | 0.50 | 1,228 | 96,961 | 963,156 | 0.02 | 43.60 | 437.93 | 113.8% | 103.5% |
| 4 | DA | 1.00 | 1,218 | 109,164 | 1,271,097 | 0.02 | 49.15 | 578.20 | 128.3% | 136.7% |
| 5 | | 2.00 | 1,276 | 113,344 | 1,178,351 | 0.05 | 51.05 | 535.95 | 133.2% | 126.7% |
| 6 | Tween 80 | 0.50 | 1,166 | 85,714 | 844,174 | 0.00 | 38.48 | 383.74 | 100.4% | 90.7% |
| 7 | | 1.00 | 1,274 | 88,615 | 828,909 | 0.05 | 39.80 | 376.79 | 103.9% | 89.1% |
| 8 | | 2.00 | 1,272 | 92,515 | 820,119 | 0.04 | 41.57 | 372.79 | 108.5% | 88.1% |
| 9 | Tween 20 | 0.50 | 1,295 | 88,824 | 832,939 | 0.05 | 39.89 | 378.63 | 104.1% | 89.5% |
| 10 | | 1.00 | 1,349 | 88,000 | 838,616 | 0.08 | 39.52 | 381.21 | 103.1% | 90.1% |
| 11 | | 2.00 | 1,237 | 89,508 | 874,132 | 0.03 | 40.20 | 397.38 | 104.9% | 93.9% |
| 12 | NP-40 | 0.50 | 1,235 | 87,175 | 811,642 | 0.03 | 39.14 | 368.93 | 102.1% | 87.2% |
| 13 | | 1.00 | 1,262 | 87,814 | 778,995 | 0.04 | 39.43 | 354.06 | 102.9% | 83.7% |
| 14 | | 2.00 | 1,316 | 93,152 | 835,527 | 0.06 | 41.86 | 379.80 | 109.2% | 89.8% |
| 15 | DA + | 1.00 | 1,308 | 105,749 | 1,226,568 | 0.06 | 47.60 | 557.91 | 124.2% | 131.9% |
| | Tween 80 | 1.00 | | | | | | | | |
| 16 | DA + | 1.00 | 1,314 | 105,095 | 1,243,449 | 0.06 | 47.30 | 565.60 | 123.4% | 133.7% |
| | Tween 20 | 1.00 | | | | | | | | |
| 17 | DA + | 1.00 | 1,225 | 109,609 | 1,209,038 | 0.02 | 49.35 | 549.93 | 128.8% | 130.0% |
| | NP-40 | 1.00 | | | | | | | | |

CA: Cholic acid
DA: Deoxycholic acid
NS: Negative specimen
LS: Low value specimen
MS: Middle value specimen

### Example 17: Destruction of Capsid by Specimen Treatment

The destruction efficiency of the capsid by specimen treatment was studied.

Here, the method was carried out such that the extracted RNA was quantified by the RT-PCR method with semi-quantification of the genome RNA that was extracted from the virus particles. In the case where the RNase is added in advance to the specimen treatment solution or to the specimen-diluting solution that is used as the control thereby destructing the virus capsid by the specimen treatment solution, the RNA is decomposed before conducting the RNA extraction treatment. By comparing with the RNA amount before conducting the RNase treatment, the RNA amount extracted from the particles by the treatment solution can be estimated. With this, the destruction efficiency of the capsid can be estimated.

Specifically, each of the specimen dilution solution (Tris buffer, 150 mM of NaCl, and 1% of BSA), the specimen treatment solution (Tris buffer, 150 mM of NaCl, 1% of BSA, and 0.1% of NaN₃) containing 0.25% of C16APS, and the specimen treatment solution containing 0.25% of C16APS and the surfactant with the concentration thereof as listed in Table 16, all of these solution being added with RNase A (final concentration of 200 μg/mL), was added into the same amount of the specimen prepared by adding the purchased nasopharyngeal wiping solution (Boca Biolistics, LLC) into the specimen dilution solution. After they were mixed, the resulting mixture was allowed to statically leave at room temperature for 5 minutes. Then, by using the RNA extraction kit (GeneJET Viral DNA/RNA Purification kit; Thermo Fisher), RNA was extracted in accordance with the guidance attached to the kit. The RNA thereby obtained was measured by using the RT-PCR kit (SARS-CoV-2 Direct Detection RT-qPCR Kit (TaKaRa)); then, the Ct value of RT-PCR was calculated. As the Ct value is more, RNA is destructed more readily, namely, the destruction efficiency of the capsid is higher. The results are listed in Table 15 and Table 16.

TABLE 15
Study about destruction efficiency of capsid (1)

| Specimen treatment | RNase | | |
|---|---|---|---|
| (RT, 5 minutes) | treatment | Ct (dRn) | Copies/rxn |
| Specimen dilution solution | − | 24.1 | 1.92E+05 |
| Specimen treatment solution | − | 24.4 | 1.58E+05 |
| (without addition) (C16APS) | | | |
| Specimen dilution solution | + | 28.8 | 7.78E+03 |
| Specimen treatment solution | + | 33.7 | 2.77E+02 |
| (without addition) (C16APS) | | | |

TABLE 16
Study about destruction efficiency of capsid (2)

| Treatment solution | Ct (dRn) | Ct mean | Copies/rxn |
|---|---|---|---|
| Specimen treatment solution | 33.2 | 32.4 | 6.00E+02 |
| (without addition) | 31.7 | | |
| (0.25% C16APS) | | | |
| +0.5% Deoxycholic acid | 31.7 | 32.5 | 5.70E+02 |
| | 33.3 | | |
| +1% Deoxycholic acid | 35.6 | 36.2 | 4.40E+01 |
| | 36.9 | | |
| +2% Deoxycholic acid | 37.7 | 38.3 | 1.06E+01 |
| | 38.8 | | |
| +1% Deoxycholic acid | 37.1 | 37.2 | 2.16E+01 |
| +Tween 20 | 37.4 | | |

As a result, when treated with the specimen treatment solution (without addition), the Ct value was increased by 4.9 against the specimen dilution solution. Namely, the RNA amount decreased to about 1/30; and it was thus found that the extraction efficiency of RNA increased 30 times.

When 0.5% of deoxycholic acid was added to the specimen treatment solution, there was no significant change in the Ct value; but when the amount thereof was increased to 1% and then to 2%, the Ct values thereof increased by 3.8 and 4.9, respectively. Namely, the RNA amounts decreased to about 1/14 and to 1/30, respectively; and thus it was found that the extraction efficiency of RNA increased 14 times and 30 times, respectively.

In addition, when the nonionic surfactant (Tween 20) was added to the specimen treatment solution, the Ct value increased by 1. Namely, the RNA amount decreased to ½, indicating that the RNA extraction efficiency was enhanced.

From these results, it became clear that C16APS is effective in extraction of the capsid, and that addition of 1% or more of deoxycholic acid and the nonionic surfactant thereto can further enhance the extraction efficiency of RNA, namely, the destruction efficiency of the capsid can be enhanced.

### EXPLANATION OF REFERENCES

1 immunochromatographic cartridge

2 Matrix

3 Detecting zone

4 Labelled reagent zone

5 Developing solution tank

6 Development confirming part

7 Developing solution absorbing zone

8 Substrate zone

9 Developing solution pad

1. A method of immunologically detecting SARS-CoV-2, the method comprising detecting a SARS-CoV-2 nucleocapsid protein (N protein) in a specimen taken from a subject by using the following antibodies (1) and (2):
(1) a first antibody to a first epitope in a 260th to 305th amino acid region in the SARS-CoV-2 N protein; and
(2) a second antibody to a second epitope in a 365th to 419th amino acid region in the SARS-CoV-2 N protein.

2. The method according to claim 1, wherein a third antibody to a third epitope in a 120th to 147th amino acid region in the SARS-CoV-2 N protein is further used.

3. The method according to claim 1, wherein a fourth antibody to a fourth epitope in a 44th to 78th amino acid region in the SARS-CoV-2 N protein is further used.

4. The method according to claim 1, wherein a fifth antibody to a fifth epitope in a 243rd to 259th amino acid region in the SARS-CoV-2 N protein is further used.

5. The method according to claim 1, wherein a sixth antibody to a sixth epitope in a 306th to 339th amino acid region in the SARS-CoV-2 N protein is further used.

6. The method according to claim 1, wherein both the first antibody and the second antibody are used as a solid phase antibody or a labelled antibody.

7. The method according to claim 6, wherein
when both the first antibody and the second antibody are used as the solid phase antibody, the third antibody is used as the labelled antibody, and
when both the first antibody and the second antibody are used as the labelled antibody, the third antibody is used as the solid phase antibody.

8. The method according to claim 6, wherein
the fourth antibody is used as the solid phase antibody and the fifth antibody is used as the labelled antibody, or
the fourth antibody is used as the labelled antibody and the fifth antibody is used as the solid phase antibody.

9. The method according to claim 1, wherein the detection is carried out by a sandwich method.

10. A reagent of immunologically detecting SARS-CoV-2, the reagent comprising the following antibodies (1) and (2):
(1) a first antibody to a first epitope in a 260th to 305th amino acid region in a SARS-CoV-2 N protein; and
(2) a second antibody to a second epitope in a 365th to 419th amino acid region in the SARS-CoV-2 N protein.

11. The reagent according to claim 10, the reagent further comprising one or more antibodies selected from the group consisting of the following (3) to (6):
(3) a third antibody to a third epitope in a 120th to 147th amino acid region in the SARS-CoV-2 N protein;
(4) a fourth antibody to a fourth epitope in a 44th to 78th amino acid region in the SARS-CoV-2 N protein;
(5) a fifth antibody to a fifth epitope in a 243rd to 259th amino acid region in the SARS-CoV-2 N protein; and
(6) a sixth antibody to a sixth epitope in a 306th to 339th amino acid region in the SARS-CoV-2 N protein.

12. The reagent according to claim 10, wherein both the first antibody and the second antibody are a solid phase antibody or a labelled antibody.

13. The reagent according to claim 10, wherein the reagent is used in a sandwich method.

14. The reagent according to claim 11, wherein the reagent comprises a solid phase.
